(19) United States
(12) Patent Application Publication
(10) Pub. No.: US 2026/0354410 A1
(43) Pub. Date: Nov. 12, 2026

(54) Edible Composition Comprising Recombinant Human Secretory Protein

(71) Applicant: Helaina Inc., New York City, NY (US)

(72) Inventors: Laura Katz, New York City, NY (US); Marie Tremblay, New York City, NY (US); Anthony Clark, New York City, NY (US); Inseob Choi, New York City, NY (US)

(73) Assignee: Helaina Inc., New York City, NY (US)

(21) Appl. No.: 19/102,810

(22) PCT Filed: Aug. 9, 2023
(86) PCT No.: PCT/US23/71955

§ 371 Date: Feb. 10, 2025

Related U.S. Application Data
(60) Provisional application No. 63/371,174, filed on Aug. 11, 2022.

(51) Int. Cl. A23J 3/08; A23C 9/13; A23C 9/152; A23C 9/158; A23G 3/44; A23G 9/38; A23J 3/10; A23J 3/14; A23J 3/16; A23L 2/66; A23L 33/00

(52) U.S. Cl. CPC A23J 3/08; A23C 9/1307; A23C 9/1522; A23C 9/1526; A23C 9/158; A23G 3/44; A23G 9/38; A23J 3/10; A23J 3/14; A23J 3/16; A23L 2/66; A23L 33/40

(57) ABSTRACT

Disclosed are recombinant human secretory proteins, along with compositions comprising such proteins and methods for using such compositions. Aspects of the disclosure include microbially generated human secretory proteins and edible and potable compositions containing same, such as, an infant formula comprising such proteins. Also disclosed are methods for use of the disclosed edible and potable compositions to provide nutrition and other benefits.

### SEQUENCE LISTING

The instant application contains a Sequence Listing submitted in xml format and hereby is incorporated by reference in entirety. Said xml copy, created on 4 Aug. 2023, is named 080201-80180201.xml and is 7,577 bytes in size.

### BACKGROUND

### I. Technical Field

Embodiments relate to human secretory proteins, compositions comprising such proteins, and use thereof in specific edible compositions, such as, food, beverage and supplement compositions, including an infant formula.

### II. Background

Secretory proteins have important roles in a body, including nutrition, protection, lubrication and so on. Dedicated cell machinery is needed to modify an incipient secreted protein, for example, glycosylation, and to traffic that nascent secretory protein to a membrane or out of a cell.

Although from a phylogenetic perspective, secretory proteins may be similar across taxa, the range, amount and structure of a secretory protein is a species-specific characteristic or feature. For example, in mammal milk, type of secretory protein, amount of a secretory protein and structure of a secretory protein, for example, nature of glycosylation and structure of carbohydrates, vary, and can vary considerably from species to species. Thus, for example, the secretory protein or glycosylation profile, pattern or fingerprint of bovine milk varies from that of human milk.

(Secretions, serous fluid and the like containing secretory proteins also contain a range of lipids and carbohydrates, which can be unique to an organ, a stage of life and so on. Hence, for example, human milk contains fat globules encased in a milk fat globule membrane (MFGM), a complex assemblage of lipids and proteins. Composition of MFGM can vary as an infant ages. Oligosaccharides (human milk oligosaccharides, HMO), also vary in content and amount amongst species. HMO's of breast milk are not necessarily an energy source for an infant, instead, may serve to provide a conducive environment and food source of microbes in the gut of an infant.)

Because of variability of secretory molecules among species, the goal of isolating and using secretory molecules for therapeutic or nutritional value in human is elusive and currently unattained as obtaining commercial amounts of collected human secretory molecules is not feasible; and species differences translate to use of non-human secretory molecules resulting in product imperfectly matched for human use. If a current commercial product lacking a molecule normally found in human milk were to contain an excess of a compensatory non-human ingredient, that can be a burden to a subject and so on.

For example, many current infant formulae are produced with food ingredients derived from conventional agricultural and manufacturing practices. In recent years, manufacturers have attempted to create formulations with components that more closely resemble human secretory protein, and for example, breast milk. Microbially-expressed HMO's are added to some branded infant formulae. Such technologies have proven to be useful in making high-value and densely nutritious food ingredients that are unattainable from conventional agricultural practices or from natural sources.

Infant formula commonly is based on bovine milk proteins, which differ from human breast milk proteins, for example, in total amino acid composition. To address amino acid needs of a human infant, bovine protein level in infant formula generally is raised. That provides a much higher total intake of protein by formula-fed infants, which can precipitate metabolic stress on infants.

Increased protein intake resulting from high protein content of formulae on the market leads to higher insulin levels in formula-fed infants. Formula-fed infants get about 70% more protein than do breastfed infants between 3-6 months of age (for example, Heinig et al., Am J Clin Nutr, 1993; 58:152-156; and Koletzko et al., Adv Exp Med Biol, 2005; 569:69-79).

Morbidity differences between breastfed and formula-fed infants have been observed, breastfed infants have fewer respiratory infection, ear infection and gastroenteritis than do formula-fed infants. (Wright et al., BMJ, 299, 946-949, 1989; Duncan et al., Pediatrics, 2003, 91(5)867-873; Aniansson et al., Pediatr Infect Dis J, 1994, 13(3)182-188; and Dewey et al., J Pediatr, 1992, 126(5)part 1:695-702). A possible explanation is greater level of immune modulating substances in human as compared to bovine milk.

For example, approximately 70% of protein found in human milk is whey protein. Primary whey proteins are α-lactalbumin, lactoferrin (LF) and secretory IgA. Those proteins are bioactive and possess unique properties, such as, antimicrobial activities, anti-inflammatory activity and anti-oxidative properties. Those proteins also may aid in absorption of key vitamins and minerals.

Human milk whey proteins for human consumption currently are unavailable for commercial use. Current infant formulae rely solely on protein sources from bovine, goat or soy. There are no commercially available human milk whey proteins available for use in edible or potable compositions. Human neonates who do not have access to human milk will be unable to reap the health benefits of human secretory molecules. Adults also will not benefit from the virtues of human secretory molecules.

Because of the numerous health and nutrition benefits of human secretory molecules, there is interest in including human secretory molecules in a range of edible or potable compositions, such as, drinks, infant formulae, bars, dairy products, beverages and so on.

### SUMMARY

The present disclosure addresses needs in the art by providing microbially expressed human secretory proteins, such as, human milk proteins, as well as unique edible or potable compositions (hereinafter, “edible compositions,”) comprising such proteins and methods of use for providing nutrition to a subject. Such compositions achieve immunomodulatory and other benefits of native human secretory proteins, previously only available using non-human sources. Aspects of the disclosure include combinations of individual ingredients to maximize immunomodulatory impact of a formulation. In aspects, compositions of the disclosure include, in addition to one or more human secretory proteins, one or more human secretory oligosaccharides (for example, 2′-fucosyllactose), a probiotic bacterium (for example, Bifidobacterium, such as, Bifidobacterium infantis), a lipid source, such as, MFGM, and so on.

Accordingly, disclosed herein, in aspects, is an edible composition comprising lactose, one or more lipids, protein, a human secretory oligosaccharide, such as, a human milk oligosaccharide (HMO), one or more minerals, one or more vitamins, a probiotic organism and a human secretory protein. In embodiments, an HMO is 2′-fucosyllactose, lacto-N-neotetraose, 3-fucosyllactose, 6′-sialyllactose, difucosyllactose or lacto-N-tetraose. In embodiments, an HMO is 2′-fucosyllactose. In embodiments, an HMO is lacto-N-neotetraose. In embodiments, an edible composition comprises one or more of high oleic sunflower oil, mid-oleic sunflower oil, canola oil, safflower oil, coconut oil, low erucic rapeseed oil, sunflower oil, ascorbyl palmitate, mixed tocopherol concentrate, Mortierella alpina oil, alga oil, soybean oil or palm oil. In embodiments, an edible composition comprises high oleic sunflower oil, coconut oil, low erucic rapeseed oil, canola oil, sunflower oil, ascorbyl palmitate, mixed tocopherol concentrate, Mortierella alpina oil and alga oil. In embodiments, one or more minerals comprise one or more of calcium, phosphorous, iodine, iron, magnesium, copper, zinc, manganese, chloride, potassium, sodium or selenium. In embodiments, an edible composition comprises calcium phosphate, calcium citrate, magnesium chloride, potassium citrate, potassium chloride, sodium chloride, copper sulfate, ferrous sulfate, manganese sulfate, zinc sulfate, sodium selenite or potassium iodide. In embodiments, one or more vitamins comprise one or more of vitamin A, vitamin D, vitamin E, vitamin K, thiamine, riboflavin, pyridoxine, niacinamide, calcium pantothenate, folic acid, pantothenic acid, vitamin B12, vitamin C, biotin, choline, inositol, and salts thereof. In embodiments, an edible composition comprises vitamin A palmitate, vitamin D3, D-α tocopherol, vitamin K1, thiamine hydrochloride, riboflavin, niacinamide, calcium pantothenate, pyridoxine hydrochloride, biotin, folic acid, cyanocobalamin, choline bitartrate, inositol and ascorbic acid.

In embodiments, an edible composition comprises one or more nucleotides. In embodiments, one or more nucleotides comprise one or more of cytidine 5′-monophosphate, uridine 5′-monophosphate, adenosine 5′-monophosphate or guanosine 5′-monophosphate. In embodiments, an edible composition comprises cytidine 5′-monophosphate, disodium uridine 5′-monophosphate, adenosine 5′-monophosphate and disodium guanosine 5′-monophosphate.

In embodiments, an edible composition comprises a whey protein-lipid concentrate, for example, an edible composition comprises from about 3.0% to about 5.0% by weight whey protein-lipid concentrate.

In embodiments, an edible composition comprises milk fat globule membrane (MFGM).

In embodiments, an edible composition comprises casein, such as, a micellar casein. In embodiments, an edible composition comprises from about 5% to about 8% by weight micellar casein.

In embodiments, an edible composition comprises from about 50% to about 55% by weight of lactose powder. In embodiments, an edible composition comprises a whole milk powder. In embodiments, an edible composition comprises from about 13% to about 18% by weight whole milk powder.

In embodiments, a probiotic organism is a Bifidobacterium. In embodiments, a Bifidobacterium is Bifidobacterium infantis.

In embodiments, an edible composition comprises from about 43% to about 70% by weight of lactose, from about 0.5% to about 6% by weight of milk fat, from about 9% to about 13% by weight of protein, from about 1.5% to about 3.0% by weight of minerals, from about 0.5% to about 2.0% by weight of vitamins, from about 0.5% to about 2.0% by weight of 2′-fucosyllactose, from about 5×10⁸ cfu/100 g to about 1×10¹¹ cfu/100 g of probiotic microbe and from about 0.02% to about 0.3% by weight of a human secretory protein.

In embodiments, an edible composition comprises from about 0.01% to about 0.06% by weight of nucleotides.

In embodiments, an edible composition comprises from about 0.06% to about 0.4% by weight of arachidonic acid (ARA).

In embodiments, an edible composition comprises from about 0.03% to about 0.3% by weight of docosahexaenoic acid (DHA).

In embodiments, an edible composition comprises from about 0.00002% to about 0.00009% by weight of lutein.

In embodiments, an edible composition comprises from about 0.025% to about 0.07% by weight of taurine.

In embodiments, an edible composition comprises from about 0.002% to about 0.015% by weight of L-carnitine.

Also disclosed, in embodiments, is an edible composition comprising lactose, one or more lipids, protein, a human milk oligosaccharide (HMO), one or more minerals, one or more vitamins, a probiotic microbe and recombinant human lactoferrin (rhLF), which can comprise one or more human-like glycans, N-glycan found in human protein. Further described, in embodiments, is an edible composition comprising a recombinant human secretory protein from a non-mammal cell, a recombinant human secretory protein comprising one or more human-like glycans, N-glycans found on human glycoproteins.

In embodiments, a human secretory protein is a recombinant protein. In embodiments, a human secretory protein is from a mammal cell. In embodiments, a human secretory protein is from a non-mammal cell. In embodiments, a human secretory protein is from a bacterium. In embodiments, a human secretory protein is from a fungus cell. In embodiments, a human secretory protein is from a yeast cell. In embodiments, a yeast cell is of a genus, Arxula, Aspergillus, Aurantiochytrium, Candida, Claviceps, Cryptococcus, Cunninghamella, Geotrichum, Hansenula, Kluyveromyces, Kodamaea, Komagataella, Leucosporidiella, Lipomyces, Mortierella, Ogataea, Pichia, Prototheca, Rhizopus, Rhodosporidium, Rhodotorula, Saccharomyces, Schizosaccharomyces, Tremella, Trichosporon, Wickerhamomyces or Yarrowia. In embodiments, a human secretory protein is secretory IgA (sIgA), human serum albumin, xanthine dehydrogenase, lactoferrin, lactoperoxidase, butyrophilin, lactadherin, adiponectin, β-casein, κ-casein, leptin, lysozyme or α-lactalbumin. In embodiments, a human secretory protein is human lactoferrin. In embodiments, a human secretory protein is a human whey protein.

In embodiments, a human secretory protein comprises a human-like glycan, that is, an N-glycan found in human glycoprotein. In embodiments, a human secretory protein comprises a hybrid N-glycan. In embodiments, a human secretory protein comprises a complex N-glycan. In embodiments, a human secretory protein comprises a bi-antennary, a tri-antennary or a tetra-antennary N-glycan. In embodiments, the human secretory protein comprises a glycan comprising sialic acid, galactose, N-acetylgalactosamine or fucose. In embodiments, a human secretory protein comprises a high-mannose N-glycan. In embodiments, a human secretory protein comprises a glycan having less than ten mannose residues. In embodiments, a human secretory protein comprises a glycan having less than four mannose residues. In embodiments, a human secretory protein comprises a glycan having four or more mannose residues.

In embodiments, a method for providing nutrition to a subject comprising feeding said subject an edible composition of interest as described herein.

Also described is a method for producing an edible composition comprising generating a mixture disclosed herein, for example, a mixture comprising a protein and a human secretory protein (for example, a recombinant human milk protein, such as, a recombinant human lactoferrin, which can have human-like glycans), and optionally including one or more of, a lactose, one or more lipids, protein, a human secretory oligosaccharide, such as, a human milk oligosaccharide (HMO), one or more minerals, one or more vitamins and a probiotic microbe.

In embodiments, an edible composition comprises a food, such as, a bar, a dairy product, such as, a yogurt or an ice cream, a nutritional powder, an infant formula powder, a candy, such as, a gummy, and so on. An edible composition of interest also comprises a potable composition, such as, a beverage, a smoothie, a shake, a ready to use infant formula, a liquid nutraceutical, a drinkable nutritional composition and so on.

In a first embodiment, the invention relates to an infant formula comprising lactose, one or more lipids, protein, a human milk oligosaccharide (HMO), one or more minerals, one or more vitamins, a probiotic microbe and a human secretory protein.

In a second embodiment, the infant formula of the first embodiment comprises secretory IgA (sIgA), human serum albumin, xanthine dehydrogenase, lactoferrin (LF), such as, human LF (hLF), lactoperoxidase, butyrophilin, lactadherin, adiponectin, β-casein, κ-casein, leptin, lysozyme or α-lactalbumin.

In a third embodiment, the human secretory protein of the second embodiment is human lactoferrin.

In a fourth embodiment, in the infant formula of the first embodiment, the human secretory protein is a human whey protein.

In a fifth embodiment, the human secretory protein of any of the formula of embodiments 1-4 comprises a hybrid N-glycan.

In a sixth embodiment, the human secretory protein of any of the formula of embodiments 1-4 comprises a complex N-glycan.

In a seventh embodiment, the human secretory protein of the 6^{th} embodiment comprising a bi-antennary, tri-antennary or tetra-antennary N-glycan.

In an eighth embodiment, the human secretory protein of any of the formula of embodiments 1-4 comprises a high-mannose N-glycan.

In a ninth embodiment, the human secretory protein of any of the formula of embodiments 1-4 comprises sialic acid, galactose, N-acetylgalactosamine, or fucose.

In a tenth embodiment, the human secretory protein of any of the formula of embodiments 1-4 comprises a glycan having less than ten mannose residues.

In an eleventh embodiment, the human secretory protein of any of the formula of embodiments 1-4 comprises a glycan having less than four mannose residues.

In a twelvth embodiment, the human secretory protein of any of the formula of embodiments 1-4 comprises a glycan having four or more mannose residues.

In a thirteenth embodiment, the formula of any of the previous embodiments wherein the HMO is 2′-fucosyllactose, lacto-N-neotetraose, 3-fucosyllactose, 6′-sialyllactose, difucosyllactose or lacto-N-tetraose.

In a fourteenth embodiment, the formula of embodiment 13 comprises 2′-fucosyllactose.

In a fifteenth embodiment, the formula of embodiment 13 comprises lacto-N-neotetraose.

In a sixteenth embodiment, the formula of any of the previous embodiments comprises one or more of high oleic sunflower oil, mid-oleic sunflower oil, safflower oil, coconut oil, canola oil, low erucic rapeseed oil, sunflower oil, ascorbyl palmitate, mixed tocopherol concentrate, Mortierella alpina oil, alga oil, soybean oil and palm oil.

In a seventeenth embodiment, the formula of the 16th embodiment comprises high oleic sunflower oil, coconut oil, canola oil, low erucic rapeseed oil, sunflower oil, ascorbyl palmitate, mixed tocopherol concentrate, Mortierella alpina oil and alga oil.

In an eighteenth embodiment, the formula of any of the previous embodiments comprises one or more of calcium, phosphorous, iodine, iron, magnesium, copper, zinc, manganese, chloride, potassium, sodium and selenium.

In a nineteenth embodiment, the formula of the 18th embodiment comprises calcium phosphate, calcium citrate, magnesium chloride, potassium citrate, potassium chloride, sodium chloride, copper sulfate, ferrous sulfate, manganese sulfate, zinc sulfate, sodium selenite and potassium iodide.

In an twentieth embodiment, the formula of any of the previous embodiments comprises one or more of vitamin A, vitamin D, vitamin E, vitamin K, thiamine, riboflavin, pyridoxine, niacinamide, calcium pantothenate, folic acid, pantothenic acid, vitamin B12, vitamin C, biotin, choline, inositol, and salts thereof.

In a twenty-first embodiment, the formula of the 20^{th} embodiment comprises vitamin A palmitate, vitamin D3, D-α-tocopherol, vitamin K1, thiamine hydrochloride, riboflavin, niacinamide, calcium pantothenate, pyridoxine hydrochloride, biotin, folic acid, cyanocobalamin, choline bitartrate, inositol and ascorbic acid.

In a twenty-second embodiment, the formula of any of the previous embodiments comprising one or more nucleotides.

In a twenty-third embodiment, the formula of the 22^{nd} embodiment where one or more nucleotides comprise cytidine 5′-monophosphate, uridine 5′-monophosphate, adenosine 5′-monophosphate and guanosine 5′-monophosphate.

In a twenty-fourth embodiment, the formula of the 23^{rd} embodiment comprises cytidine 5′-monophosphate, disodium uridine 5′-monophosphate, adenosine 5′-monophosphate and disodium guanosine 5′-monophosphate.

In a twenty-fifth embodiment, the formula of any of the previous embodiments comprising whey protein-lipid concentrate.

In a twenty-sixth embodiment, the formula of the 25th embodiment comprising from about 3.0% to about 5.0% whey protein-lipid concentrate.

In a twenty-seventh embodiment, the formula of any of the previous embodiments comprising milk fat globule membrane (MFGM).

In a twenty-eighth embodiment, the formula of any of the previous embodiments comprising casein.

In a twenty-ninth embodiment, the formula of the 28th embodiment comprising from about 5% to about 8% micellar casein.

In a thirtieth embodiment, the formula of the 25^{th} to 29^{th} embodiments comprises from about 50% to about 55% by weight of lactose powder.

In a thirty-first embodiment, the formula of the 1^{st} to 24^{th} embodiments comprises whole milk powder.

In a thirty-second embodiment, the formula of any of the prior embodiments comprising from about 13% to about 18% whole milk powder.

In a thirty-third embodiment, the formula of any of the previous embodiments comprising a Bifidobacterium.

In a thirty-fourth embodiment, the formula of the 33^{rd} embodiment comprising Bifidobacterium infantis.

In a thirty-fifth embodiment, the human secretory protein of any of the formula of the previous embodiments which is a recombinant protein.

In a thirty-sixth embodiment, the human secretory protein of the formula of the 35^{th} embodiment which is from a mammal cell.

In a thirty-seventh embodiment, the human secretory protein of the formula of the 35^{th} embodiment which is from a non-mammal cell.

In a thirty-eighth embodiment, the human secretory protein of the formula of the 35^{th} embodiment which is from a bacterium.

In a thirty-ninth embodiment, the human secretory protein of the formula of the 35^{th} embodiment which is from a fungus cell.

In a fortieth embodiment, the human secretory protein of the formula of the 35^{th} embodiment which is from a yeast cell.

In a forty-first embodiment, the human secretory protein of the formula of the 40^{th} embodiment which is from a yeast cell of an Arxula, Aspergillus, Aurantiochytrium, Candida, Claviceps, Cryptococcus, Cunninghamella, Geotrichum, Hansenula, Kluyveromyces, Kodamaea, Komagataella, Leucosporidiella, Lipomyces, Mortierella, Ogataea, Pichia, Prototheca, Rhizopus, Rhodosporidium, Rhodotorula, Saccharomyces, Schizosaccharomyces, Tremella, Trichosporon, Wickerhamomyces or Yarrowia.

In a forty-second embodiment, the formula of any the prior embodiments comprises from about 43% to about 70% by weight of lactose, from about 0.5% to about 6% by weight of milk fat, from about 9% to about 13% by weight of protein, from about 1.5% to about 3.0% by weight of minerals, from about 0.5% to about 2.0% by weight of vitamins, from about 0.5% to about 2.0% by weight of 2′-fucosyllactose, from about 5×10⁸ cfu/100 g to about 1×10¹¹ cfu/100 g of the probiotic organism and from about 0.02% to about 0.3% by weight of the human milk protein.

In a forty-third embodiment, the formula of any of the previous embodiments comprising from about 0.01% to about 0.06% by weight of nucleotides.

In a forty-fourth embodiment, the formula of any of the previous embodiments comprising from about 0.06% to about 0.4% by weight of arachidonic acid.

In a forty-fifth embodiment, the formula of any of the previous embodiments comprising from about 0.03% to about 0.3% by weight of docosahexaenoic acid.

In a forty-sixth embodiment, the formula of any of the previous embodiments comprising from about 0.00002% to about 0.00009% by weight of lutein.

In a forty-seventh embodiment, the formula of any of the previous embodiments comprising from about 0.025% to about 0.07% by weight of taurine.

In a forty-eighth embodiment, the formula of any of the previous embodiments comprising from about 0.002% to about 0.015% by weight of L-carnitine.

The forty-ninth embodiment relates to an infant formula comprising lactose, one or more lipids, protein, a human milk oligosaccharide (HMO), one or more minerals, one or more vitamins, a probiotic microbe, and a recombinant human lactoferrin protein comprising one or more human-like glycans.

In a fiftieth embodiment, the formula of the 49^{th} embodiment wherein the recombinant human lactoferrin protein is from a non-mammal cell.

In a fifty-first embodiment, the formula of the 49^{th} or 50th embodiment wherein the recombinant human lactoferrin protein is from a yeast cell.

The fifty-second embodiment relates to a method for providing nutrition to an infant comprising feeding to an infant the infant formula of any of the prior embodiments.

The fifth-third embodiment relates to an infant formula comprising a recombinant human secretory protein from a non-mammal cell, comprising one or more human-like glycans.

In a fifty-fourth embodiment, in the infant formula of the 53rd embodiment, the human secretory protein is secretory IgA (sIgA), human serum albumin, xanthine dehydrogenase, lactoferrin, lactoperoxidase, butyrophilin, lactadherin, adiponectin, β-casein, κ-casein, leptin, lysozyme, or α-lactalbumin.

In a fifty-fifth embodiment, in the infant formula of the 53rd embodiment, the human secretory protein is human lactoferrin (hLF).

In a fifty-sixth embodiment, in the infant formula of the 53rd embodiment, the human secretory protein is a human whey protein.

In a fifty-seventh embodiment, the human secretory protein of embodiments 53-56 comprising a glycan comprising sialic acid, galactose, N-acetylgalactosamine, or fucose.

In a fifty-eighth embodiment, the human secretory protein of embodiments 53-56 comprises a complex N-glycan.

In a fifth-ninth embodiment, the human secretory protein of embodiment 58 comprises a bi-antennary, tri-antennary or tetra-antennary N-glycan.

In a sixtieth embodiment, the human secretory protein of embodiments 53-56 comprising a hybrid N-glycan.

In a sixty-first embodiment, the human secretory protein of the 53rd embodiment is from a bacterium.

In a sixty-second embodiment, the human secretory protein of the 53rd embodiment is from a fungus cell.

In a sixty-third embodiment, the human secretory protein of the 53rd embodiment is from a yeast cell.

In a sixty-fourth embodiment, the yeast cell of the 63rd embodiment is of a genus Arxula, Aspergillus, Aurantiochytrium, Candida, Claviceps, Cryptococcus, Cunninghamella, Geotrichum, Hansenula, Kluyveromyces, Kodamaea, Komagataella, Leucosporidiella, Lipomyces, Mortierella, Ogataea, Pichia, Prototheca, Rhizopus, Rhodosporidium, Rhodotorula, Saccharomyces, Schizosaccharomyces, Tremella, Trichosporon, Wickerhamomyces or Yarrowia.

The sixty-fifth embodiment relates to a method for providing nutrition to an infant comprising feeding to an infant the infant formula of any of embodiments 53-64.

The sixty-sixth embodiment relates to a method for producing an infant formula comprising generating a mixture comprising lactose, one or more lipids, bovine protein, a human milk oligosaccharide (HMO), one or more minerals, one or more vitamins, a probiotic microbe, and a human secretory protein.

In a sixty-seventh embodiment, in the method of the 66^{th} embodiment, the human secretory protein is from a mammal cell.

In a sixty-eighth embodiment, in the method of the 66^{th} embodiment, the human secretory protein is from a non-mammal cell.

In a sixty-ninth embodiment, in the method of the 68th embodiment, the cell is a yeast cell.

In a seventieth embodiment, in the method of the 69th embodiment, the yeast cell is of a genus of Arxula, Aspergillus, Aurantiochytrium, Candida, Claviceps, Cryptococcus, Cunninghamella, Geotrichum, Hansenula, Kluyveromyces, Kodamaea, Komagataella, Leucosporidiella, Lipomyces, Mortierella, Ogataea, Pichia, Prototheca, Rhizopus, Rhodosporidium, Rhodotorula, Saccharomyces, Schizosaccharomyces, Tremella, Trichosporon, Wickerhamomyces or Yarrowia.

In a seventy-first embodiment, in the method of embodiments 66-70, the human secretory protein is secretory IgA (sIgA), human serum albumin, xanthine dehydrogenase, lactoferrin, lactoperoxidase, butyrophilin, lactadherin, adiponectin, β-casein, κ-casein, leptin, lysozyme or α-lactalbumin.

In a seventy-second embodiment, in the method of the 71st embodiment, the human secretory protein is human lactoferrin.

In a seventy-third embodiment, in the method of the 66^{th}-70^{th} embodiments, the human secretory protein is human whey protein.

In a seventy-fourth embodiment, in the method of the 66^{th}-73rd embodiments, the human secretory protein comprises a glycan comprising sialic acid, fucose, galactose or N-acetylgalactosamine.

In a seventy-fifth embodiment, in the method of the 74th embodiment, the human secretory protein comprises a complex N-glycan.

In a seventy-sixth embodiment, in the method of the 75th embodiment, the human secretory protein comprises a bi-antennary, tri-antennary or tetra-antennary N-glycan.

In a seventy-seventh embodiment, in the method of the 66^{th}-73rd embodiments, the human secretory protein comprises a hybrid N-glycan.

In a seventy-eighth embodiment, in the method of the 66^{th}-73rd embodiments, the human secretory protein comprises a high-mannose N-glycan.

It is contemplated that any embodiment discussed herein can be implemented with respect to any method or edible composition of embodiments of the disclosure, and vice versa. Furthermore, compositions of certain embodiments can be used to achieve methods of certain embodiments.

Other objects, features and advantages of the present disclosure will become apparent from the following detailed description. It should be understood, however, that the detailed description and the specific examples, while indicating specific embodiments of the disclosure, are given by way of illustration only, since various changes and modifications will become apparent to those skilled in the art from the instant detailed description.

### DETAILED DESCRIPTION

Aspects of the present disclosure address the lack or scarcity of human secretory proteins in the commercial market by including a source of recombinant human secretory proteins (for example, lactoferrin) for use as a food ingredient or foodstuff in edible compositions, such as, an infant formula. That will bring the antimicrobial, anti-inflammatory and anti-oxidative properties of those proteins to consumers who previously, realistically, could only access those proteins in non-human milk, despite interspecies differences in milk of different mammals, or from human milk. Edible compositions comprise, in addition to one or more human secretory protein, custom combinations of other individual ingredients of similar biologic activity to maximize immunomodulatory and nutritional impact of a formulation of interest. Also disclosed is an edible composition comprising one or more human secretory proteins together with one or more human oligosaccharides, such as, human milk oligosaccharides (HMO) with probiotic microbes (for example, a Bifidobacterium, such as, Bifidobacterium infantis).

### I. DEFINITIONS

An, “infant formula,” is any composition comprising components which serve as a simulation of human milk or as a complete or partial substitute for human milk. In aspects, an, “infant formula,” is a composition as defined in 21 U.S.C. § 321. An infant formula of the present disclosure may be a solid composition (for example, a powder). An infant formula of the disclosure may be a liquid composition. A liquid composition can be a concentrate or ready to use.

A, “nutritional composition,” or an, “edible composition,” according to the instant invention is, for example, an, “infant formula,” and relates to a foodstuff intended for nutritional use by infants from birth and during the first 6-12 months of life and satisfying nutritional requirements of infants and babies. An infant formula can be intended for use as the only source of nutrients from birth to about 6 months of age. A formula used in the first six months of life can be termed a, “starter formula.”

The term, “preterm infant formula,” means an infant formula intended for a preterm infant.

A, “milk fortifier,” refers to liquid or solid edible and nutritional compositions suitable for mixing with breast milk (which is human milk for a human milk fortifier) or infant formula. A milk fortifier can be used to increase calories, protein content, mineral amount and vitamins in breast milk fed to preterm infants or infants with a low birth weight. “Breast milk,” is not only milk but also relates to colostrum of a mother, or a donor milk or a colostrum of a donor.

Further, an edible composition according to the invention may be intended as a, “follow-on formula,” a complement or as a part of a progressively diversified diet wherein formula feeding starts, for example, when an infant is about 4 months of age and feeding of formula is part of an infant diet up to the first year birthday of a baby. An infant may start to include a formula according to the invention between 4-6 months, which is when most infants start with solids, or at least when breastfed infants nutrient intake is complemented with solids from 6 months up to 12 months. A follow-on formula is a formula intended to be used from about 6 months onwards during a weaning period, as a complement to solids which are introduced at about 6 months.

An infant formula can be mixed with a cereal to form an, “infant cereal composition,” a foodstuff intended for particular nutritional use by infants or children, such as, young children, during first years of life.

An edible composition can be a powder concentrate or a liquid concentrate intended to be mixed with water before use, or a liquid ready-to-use product.

The term, “ready-to-use product,” or synonymous equivalent terms, such as, ready to feed, ready to drink or ready to consume, as used herein, unless otherwise specified, refers to liquid formulae suitable for direct oral administration out of a packaging to an infant, wherein a formula is a ready to-feed liquid, a reconstituted powder or a diluted liquid concentrate.

As used herein, an, “infant,” is a child under the age of 12 months.

“Isolated,” is used to describe a biologic moiety that is removed from a normal environment thereof. Isolating is a form of, “purifying,” as extraneous material is removed from a target of interest. Thus, a gene can be excised from a chromosome away from upstream and downstream elements normally associated with an open reading frame. Similarly, a protein can be removed from the soup of the intracellular milieu or can be dissociated from an intracellular structure or from a membrane, for example. An external intervention removes an entity from normal surroundings thereof to isolate that entity.

“Recombinant,” in biotechnology or molecular biology means pieces of biologic molecules not normally found together are joined to form a new, artificial, synthetic composite molecule. Hence, pieces of nucleic acids of different origin are joined to make a new, unnatural unitary nucleic acid that otherwise, would not be found in nature. Similarly, as to proteins, polypeptides or domains thereof can be joined to form a new molecular entity with composite or new functions, that otherwise would not be found in nature. A recombinant is artificial. A synonymous term in the art is, “engineered,” or grammatic forms thereof.

An, “N-glycan,” is an N-linked oligosaccharide, that is, a sugar attached by an asparagine-N-acetylglucosamine linkage to nitrogen of an asparagine residue of a polypeptide.

A, “hybrid,” N-glycan is a glycan (also termed a polysaccharide) having both substituted (GlcNAc linkage) and unsubstituted mannose residues.

A, “complex,” N-glycan is a glycan having at least one GlcNAc attached to a 1,3-mannose arm and at least one GlcNAc attached to a 1,6-mannose arm of a trimannose core. In cases, a complex N-glycan has at least one branch that terminates in an oligosaccharide such as, for example, NeuNAc-, NeuAca2-6GalNAca1-, NeuAca2-3Galb1-3GalNAca1- or NeuAca2-3/6Galb1-4GlcNAcb1-. Complex N-glycans also can have intrachain substitutions comprising, “bisecting,” GlcNAc and core fucose (“Fuc”).

A, “high-mannose,” glycan is a glycan having at least 4 mannose residues. In aspects, a high-mannose glycan has 4, 5, 6, 7, 8, 9 or more mannose (Man) residues.

“Milk fat globule membrane,” (MFGM) comprises about 120 different proteins in a phospholipid double layer surrounding fat droplets in milk. Sphingomyelin, phosphatidyl choline, phosphatidyl serine and phosphatidyl ethanolamine are dominating phospholipids of MFGM. Butyrophilin, MUC1, PAS6/7 (lactadherin), CD14, TLR1 and TLR4 are examples of MFGM proteins, those with antimicrobial effect (Spitsberg, J Dairy Sci, 2005, 88:2289-2294; Reinhardt & Lippolis, J Dairy Res, 2006,. 73(4)406-416; Brink & Lonnerdahl, J Nutr Biochem, 86:108465, 2020; Wang et al., Fron Nutr 8:807284, 2022; and Chai et al., Food Sci Anim Res 42(3)351-371, 2022).

Lacprodan MFGM-10 (Arla Foods, Viby J, Denmark) or similar raw materials from other suppliers are enriched in MFGM and may be used as enriched phospholipid whey protein concentrate solids or a lipid source in an edible composition of interest.

“Recombinant,” as introduced above, refers to a cell, nucleic acid, protein or vector, which has been modified due to introduction of an exogenous nucleic acid or alteration of a native nucleic acid by human manipulation or intervention. Resulting cells, nucleic acids, proteins or vectors are considered recombinant, as are progeny, offspring, duplications or replications thereof, which also are considered recombinant. Thus, for example, recombinant cells can express genes not found within a native (non-recombinant) form of the cell or express native genes differently than those same genes are expressed by a non-recombinant cell. Recombinant cells can, without limitation, include recombinant nucleic acids that encode for a gene product or for suppression elements, such as mutations, knockouts, antisense, interfering RNA (RNAi) or dsRNA and so on that reduce level of an active gene product in a cell. A, “recombinant nucleic acid,” is derived from nucleic acid originally formed in vitro, in general, by exogenous, human-originated or human-mediated manipulation of nucleic acid, for example, using polymerases, ligases, exonucleases and endonucleases, or otherwise to form an artificial form not normally found in nature. Once a recombinant nucleic acid is made and introduced into a host cell or organism, that artificial, synthetically created nucleic acid may replicate using the in vivo cellular machinery of a host cell; however, such nucleic acids, once produced recombinantly, although subsequently replicated intracellularly, are still considered recombinant for purposes of the instant disclosure. Additionally, a recombinant nucleic acid refers to nucleotide sequences that comprise an endogenous nucleotide sequence and an exogenous nucleotide sequence; thus, an endogenous gene that has undergone recombination with an exogenous promoter is a recombinant nucleic acid. A, “recombinant protein,” is a protein made using recombinant techniques, for example, through expression of a recombinant nucleic acid.

From perspective of a consumer of an edible composition of interest, aside from age, “individual,” “infant,” “subject,” and, “patient,” are used interchangeably herein, and can refer to a human or a non-human.

Throughout the instant application, “about,” is used to indicate a value includes inherent variation of error of a measurement or quantification method. Such variation can be up to 10% above and below a value, that is, (0.9×q) to q to (1.1×q).

Use of, “a,” or, “an.” may mean “one,” but also is consistent with, “one or more,” “at least one,” and, “one or more than one.”

“And/or,” means, “and,” or, “or.” To illustrate, A, B, and/or C includes: A alone, B alone, C alone, a combination of A and B, a combination of A and C, a combination of B and C, or a combination of A, B, and C. In other words, “and/or,” operates as an inclusive or.

“Comprising,” (and any grammatic form of comprising, such as, “comprise,” and, “comprises,”) “having,” (and any grammatic form of having, such as, “have,” and, “has,”) “including” (and any grammatic form of including, such as, “includes,” and, “include,”) or “containing” (and any grammatic form of containing, such as, “contains,” and, “contain,”) are inclusive or open-ended and do not exclude additional, unrecited elements or method steps.

Compositions and methods for use thereof can, “comprise,” “consist essentially of,” or, “consist of,” any of ingredients or steps disclosed throughout the specification. Compositions and methods, “consisting essentially of,” ingredients or steps disclosed defines scope of a claim to specified materials or steps which materially impact the basic and novel characteristic of the claimed embodiment.

“Foodstuff,” a focus of the instant invention, relates to a composition that is consumed. Consumable foodstuffs include compositions that are edible or are used to produce foodstuffs, that is, food-grade ingredients that are edible.

“Food grade,” relates to material used to make a foodstuff or is permitted to come into direct contact with a foodstuff intended for human consumption. Food grade generally means a material that is FDA compliant and meets FDA guidelines and rules for foodstuffs. Such FDA guidelines and rules define when a foodstuff is generally regarded as safe (GRAS) for consumption.

Foodstuffs can be manufactured and handled under local and federal guidelines and regulations, such as, compliance with ISO 9001 or 22000 (International Organization of Standardization, Geneva, CH) and GMP guidelines provided by the FDA, such as, 21 CFR, various parts, such as, 117, to provide foodstuffs safe for human consumption.

As used herein, “edible,” relates to a composition that can be consumed by a subject. An edible composition can be a solid or a liquid. As known, a solid has definite volume and definite shape, such as, a powder, a bar, a gummy, a pasta and so on. A liquid, a potable, has volume but no shape, such as, a beverage, a syrup, a lotion, a tonic and so on.

Edible food products can be consumer packaged good (CPG), loose or bulk items and so on, consumable items and packages thereof found in, for example, groceries, stores, supermarkets, convenience stores, service stations, markets, vending machines and so on that vend, sell or distribute edible compositions.

As used herein, a, “flavorant,” is any ingredient in an edible composition that imparts a flavor, taste on or to that composition. A flavorant can include compounds and compositions in the food and pharmaceutic arts for imparting a taste or flavor, such as, an essence, a compound, for example, butyric acid, and so on, with no apparent or necessary nutritional value, but as used herein, flavorant also includes any ingredient, compound or composition used in an edible composition of interest to impart a flavor or taste on or to an edible composition, such as, a juice, milk, milk solids, ascorbic acid, corn syrup, lactose, sugar, nuts, which may be ground, fruit, which may be dried, macerated and so on, roots, vegetables and so on.

“Pichia pastoris,” is a yeast commonly used for commercial production of recombinant protein. Recently, the genus, Pichia, was renamed, “Komagataella,” and the species as, “Komagataella pastoris.” Other studies revealed many strains, cultures, lines and the like used in laboratories around the world identified as P. pastoris, comprise at least two species, K. pastoris and K. phaffii. For purposes herein, because the genetic differences between K. pastoris and K. phaffii are minimal and de minimis; phenotypically, the strains may not be readily distinguishable; and for all intents and purposes are substantially equivalent, despite the nomenclature, P. pastoris, K. pastoris and K. phaffii are considered equivalent, and unless specifically noted herein as specific for just one species, one name can be used for another, and use of one name includes the other two names as well. One name is substitutable for both of the other of the two names.

“Probiotic,” describes microbial cells or components of microbial cells with a beneficial effect on health or well-being of a host. A probiotic bacterium can be of the genera, Lactobacillus, Bifidobacterium and Bacillus, known to possess probiotic activity. A probiotic composition can comprise a fungus or a yeast.

Probiotic microbes may be present in a compositions of any aspect or embodiment of the instant invention, for example, in an amount: ≥5 million, ≥10 million, ≥15 million, ≥20 million, ≥25 million, ≥30 million, ≥35 million, ≥45 million, ≥50 million, ≥75 million, ≥100 million, ≥250 million, ≥500 million, ≥750 million, ≥1 billion or ≥2 billion bacteria per serving. For example, a probiotic may be present in amounts of: 5 million to 2500 million, 10 million to 2500 million, 30 million to 2500 million, 50 million to 2500 million, 50 million to 1000 million, 75 million to 2500 million, 75 million to 1000 million, 100 million to 2500 million, 100 million to 1000 million, 250 million to 2500 million, 250 million to 1000 million, 500 million to 2500 million, 500 million to 1000 million, 750 million to 2500 million or 750 million to 1000 million, 1 billion to 2.5 billion, 1.5 to 2.5 billion or more microbes per serving.

Compositions of the instant invention may be formulated to provide a daily dose of a probiotic microbe of, for example, from 10³ to 10¹⁴, 10⁴ to 10¹², 10⁵ to 10¹², 10⁶ to 10¹², 10⁷ to 10¹¹, from about 10⁷ to about 10¹⁰ or more colony forming units (cfu), microbes or other unit of measurement.

A probiotic microbe may be cultured according to any suitable method and prepared for addition to an edible composition by known techniques, such as, for example, freeze-drying or spray-drying. Probiotic microbe preparations can be purchased, for example, from Morinaga Milk, Institut Rosell, Christian Hansen and so on.

A, “prebiotic,” molecule or moiety is a compound that fosters, encourages, supports, enhances and so on growth of beneficial microbes in vivo, such as, in the gut, providing a conducive microbiota. Generally, a prebiotic moiety is indigestible in stomach, is fermented by microbes and stimulates growth of microbes. Compounds considered prebiotic include fiber, β-glucan, inulin, oligosaccharides, pectin, HMO's, fructans, xylans and so on.

Ingredients for making an edible composition of interest are known, are available commercially as food grade or pharmaceutical grade ingredients, or can be made or obtained as taught in the art. For example, some ingredients taught herein can be purchased from Hilmar Cheese, Hilmar, CA (lactose, whey protein isolate, whey protein hydrosylate, whey protein concentrate and milk protein isolate), Foremost Farms, Middleton, WI (lactose), Adams Group, Arbuckle, CA (vegetable oil blend and lecithin), AAK USA Inc., Edison, NJ (Sn2 palmitate and vegetable oil blend), Fonterra, Chicago, IL (whole milk powder and milk protein isolate), Leprino Nutrition, Denver, CO (whey protein concentrate), Arla Foods, Basking Ridge, NJ (MFGM and α-lactalbumin enriched whey protein), DSM Foods USA, Germantown, WI (mineral mix, 2′-fucosyllactose, lutein, DHA oil and ARA-DHA blend), Balchem, Montvale, NJ (choline bitartrate), Univar, Baltimore, MD (potassium bicarbonate), Chr. Hansen, New Berlin, WI (2′-fucosyllacrose and probiotics), Morinaga Milk, Irvine, CA (probiotics), Ingredion, Westchester, IL (corn syrup, resistant starch, pea protein concentrate and maltodextrin), ADM, Decatur, IL (soy protein isolate, flavorants, fructose and sodium citrate), DuPont, Wilmington, DE (carrageenan and cellulose), Idaho Milk Products, Jerome, ID (casein and milk protein concentrate), Sensus, Rye Brook, NJ (fructooligosaccharides), Beneo, Troy, NJ (fructooligosaccharides), ICL Food Specialties, Creve Coeur, ID (tricalcium phosphate), Lipotech, S.A., Lewisville, TX (magnesium chloride), Cargill, Minneapolis, MN (lecithin, canola oil, prebiotics, citric acid, caramel and potassium chloride), Jungbunzlauer, Des Plaines, IL (sodium citrate, zinc citrate and potassium citrate), Belle Chemical, Belle, WV (sodium hydroxide), Spectrum Chemical, Brunswick, NJ (magnesium phosphate, high oleic safflower oil and potassium phosphate), Prinova, Hanover Park, IL (flavorants), CellMark Ingredients, Naugatuck, RI (choline chloride, vitamin A, taurine and carnitine), CP Kelco, Atlanta, GA (gellan gum), Univar, Baltimore, MD (potassium iodide), Milkfood Ltd, Punjab, India (milk acid casein), Farbest, Park Ridge, NJ (fructose and vitamin D), Penta Mfg. Co., Livingston, NJ (m-inositol, tapioca starch, casein and maltitol), Foodchem Intl Coop, City of Industry, CA (maltitol, carnitine, sucralose, cocoa powder and m-inositol), Northwest Naturals, Bothell, WA (juices), Saputo, Montreal, Canada (skim milk powder), Virginia Date, Brooklyn, NY (flavorants), Firmenich, Plainsboro, NJ (flavorants), Exberry, Mierlo, Netherlands (colorants), United Sugars, Edina, MN (sugar), Graham Chem Corp, Barrington, IL (sucralose and sugar alcohol), Vyse, Schiller Park, IL (gelatin), Wilmar, Pearland TX (glucose syrup), Georg Lemke, Berlin, Germany (almond), Royal Ridge Fruits, Royal City, WA (dried fruits), Orient Resources Co, Hong Kong (pumpkin seeds), Golden Barrel, Honey Brook, PA (inverted sugar), AG Commodities, Oxford, PA (brown rice syrup and glycerin), Martin Bauer, Secaucus, NJ (fava bean protein concentrate), Puratos, Pennsauken, NJ (chocolate liquor) and so on.

### II. PROTEINS

“Protein,” or, “polypeptide,” refers to a molecule comprising at least five amino acid residues. “Wild-type,” refers to an endogenous version of a molecule that occurs naturally in an organism in the wild. A wild type form generally is the more prevalent from in a panmictic population. A common form may be, for example, a gene, a protein, a trait and so on.

In embodiments, a wild-type version of a protein or polypeptide is employed, however, in embodiments of the disclosure, a modified protein or polypeptide can be employed. The terms described above may be used interchangeably. A, “modified protein,” or, “modified polypeptide,” “mutant,” “altered,” or a, “variant,” refers to a protein or polypeptide where the chemical structure thereof, particularly the amino acid sequence thereof, is altered relative to the wild-type protein or polypeptide; or a variant carries one or more other elements attached to a wild type polypeptide. In embodiments, a modified/variant protein or polypeptide has at least one modified activity or function (recognizing that proteins or polypeptides may have multiple activities or functions). A modified/variant protein or polypeptide may be altered with respect to one activity or function yet retain a wild-type activity or function in other respects.

Where a protein specifically is mentioned herein, that reference is to a native (wild-type) or a recombinant (modified) protein, such as, an incipient secreted protein in which any signal sequence attached thereto is removed. A protein may be isolated directly from an organism of which it is native, produced by a recombinant DNA/exogenous expression method, produced by solid-phase peptide synthesis (SPPS) or other in vitro method. In embodiments, there are isolated nucleic acid segments and recombinant vectors incorporating nucleic acid sequences that encode a polypeptide.

In embodiments, size of a protein or polypeptide (wild-type or modified) may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 amino acid residues or greater, and any range derivable therein, or derivative of a corresponding amino sequence described or referenced herein. It is contemplated that polypeptides may be mutated by truncation, rendering a polypeptide shorter than a corresponding wild-type or modified form. Also, a polypeptide might be altered by fusing or conjugating a heterologous protein or polypeptide sequence with a particular function (for example, for targeting or localization, for purification etc.) thereto or therewith.

As used herein, “domain,” as known in the art, refers to a distinct functional or structural unit or portion of a protein or polypeptide, and generally refers to a sequence of amino acids with one of a structure or function recognizable by one skilled in the art associated with a protein with that one or multiple structures or functions.

“Polynucleotide,” refers to a nucleic acid molecule that either is recombinant or has been isolated from total genomic nucleic acid. Included within the term, “polynucleotide,” are oligonucleotides (nucleic acids 100 residues or less in length), recombinant vectors, including, for example, plasmids, cosmids, phage, viruses and the like. Polynucleotides include regulatory sequences, isolated substantially away from associated or related naturally occurring genes or protein encoding sequences, such as, control sequences. Polynucleotides may be single-stranded (coding or antisense) or double-stranded, and may be RNA, DNA (genomic, cDNA, synthetic and so on), analogs thereof or a combination thereof. Additional coding or non-coding sequences may, but need not, be present within a polynucleotide. A polynucleotide may be modified as known in the art for a desired purpose, such as, codon optimization, stability and so on.

“Gene,” “polynucleotide,” or, “nucleic acid,” is used to refer to a nucleic acid that encodes a protein, polypeptide or peptide (including any sequences, such as control sequences, required for proper transcription, post-translational modification or localization). As will be understood by those in the art, the term encompasses genomic sequences, expression cassettes, cDNA sequences and smaller engineered nucleic acid segments that express, or may be adapted to express, proteins, polypeptides, domains, peptides, fusion proteins and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations, having slightly different nucleic acid sequences but, nonetheless, encode the same or substantially similar protein, and so, herein, which can be identified as modified polynucleotides.

In embodiments, there are polynucleotide variants having substantial identity to sequences disclosed herein; those comprising at least 70%, 75%, 80%, 85%, 90%, 95%, 96%, 97%, 98%, or 99% or higher sequence identity, including all values and ranges there between, compared to a polynucleotide sequence provided herein using methods described herein (for example, BLAST analysis using standard parameters). In aspects, an isolated polynucleotide will comprise a nucleotide sequence encoding a polypeptide that has at least 90%, and in some cases, 95% or more identity to an amino acid sequence described herein, over the entire length of the sequence; or a nucleotide sequence complementary to said isolated polynucleotide.

Nucleic acid segments, regardless of length of the coding sequence, an open reading frame, may be combined with other nucleic acid sequences, such as, promoters, polyadenylation signals, additional restriction enzyme sites, multiple cloning sites, other coding segments other control and expression regions and the like, such that overall length may vary considerably. Nucleic acids can be any length. A nucleic acid of interest can be, for example, 5, 10, 15, 20, 25, 30, 35, 40, 45, 50, 75, 100, 125, 175, 200, 250, 300, 350, 400, 450, 500, 750, 1000, 1500, 3000, 5000 or more nucleotides in length, and/or can comprise one or more additional sequences, for example, regulatory sequences, and/or be a part of a larger nucleic acid, for example, a vector. It therefore is contemplated that a nucleic acid fragment of almost any length may be employed, with total length preferably being limited by ease of preparation and manipulation, and use in an intended recombinant nucleic acid protocol. In cases, a nucleic acid sequence may encode a polypeptide sequence with additional heterologous coding sequences, for example, to allow for purification of a polypeptide, transport, secretion, post-translational modification, or for therapeutic benefit, such as, targeting or efficacy. As discussed above, a tag or other heterologous polypeptide may be added to a modified polypeptide-encoding sequence, wherein, “heterologous,” refers to a polypeptide that is not the same as the modified polypeptide.

Polypeptides, proteins or polynucleotides encoding such polypeptides or proteins of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids, or nucleic acid substitutions; or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical to or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids or nucleic acids, or any range derivable therein, for example, of SEQ ID NOs:1-4.

Nucleotide as well as a protein, polypeptide and peptide sequences for various genes have been previously disclosed, and may be found in recognized computerized databases. Two commonly used databases are Genbank and Genpept of the National Center for Biotechnology Information (ncbi.nlm.nih.gov) and The Universal Protein Resource (UniProt at uniprot.org). Coding regions for genes may be amplified and/or expressed using techniques disclosed herein or as would be known to those of ordinary skill in the art.

It is contemplated that in compositions of the disclosure, there is between about 0.001 mg and about 10 mg of total polypeptide, peptide and/or protein per ml. Concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

### A. Human Milk Proteins

Aspects of the present disclosure include human milk proteins, as well as compositions comprising such proteins and methods of use thereof. As used herein, a, “human milk protein,” describes any protein present in human breast milk. A human milk protein includes a protein derived from (for example, isolated from) human breast milk, as well as any protein produced by other means (for example, recombinant expression, chemical synthesis etc.) having an amino acid sequence of a protein present in human breast milk, the wild type form. Various human milk proteins are recognized in the art and are contemplated herein, including, but not limited to, secretory IgA (sIgA), human serum albumin, xanthine dehydrogenase, lactoferrin (LF), lactoperoxidase, butyrophilin, lactadherin, adiponectin, β-casein, κ-casein, leptin, lysozyme and α-lactalbumin. In embodiments, a human milk protein of the disclosure is a human whey protein. In embodiments, a human milk protein of the disclosure is a recombinant human milk protein (for example, produced by a non-mammal cell, such as, a yeast cell).

Certain aspects of the disclosure are directed to human milk proteins having, “human-like,” glycans. Human-like glycans (also, “human-like glycan structures,”) are glycans having structures present in human glycoprotein. Such glycans include, for example, hybrid N-glycans, complex N-glycans, bi-antennary, tri-antennary and tetra-antennary N-glycans, high-mannose glycans and glycans comprising sialic acid, galactose, N-acetylgalactosamine or fucose. Human-like glycans include those having a Man3GlcNAc2 core structure. Accordingly, human milk proteins of the disclosure include those having one or more human-like glycans, for example, hybrid N-glycans, complex N-glycans, high-mannose glycans, bi-antennary N-glycans, tri-antennary N-glycans, tetra-antennary N-glycans and combinations thereof.

Accordingly, in embodiments, disclosed are recombinant (r) human (rh) milk proteins (for example, recombinant human lactoferrin, rhLF) comprising one or more human-like glycans. Such recombinant protein includes, for example, those produced by engineered mammal, fungus, yeast, bacterium or other cells, including engineered cells described herein. In aspects, such recombinant proteins can have a glycan pattern different from a glycan pattern of a corresponding natural or naturally occurring wild type human milk protein. For example, in embodiments, disclosed is a recombinant human lactoferrin comprising one or more human-like glycans, where the lactoferrin has a glycan pattern that can be different from a glycan pattern of any naturally occurring human lactoferrin (for example, human lactoferrin in human breast milk). Also disclosed is a composition comprising a plurality of recombinant human lactoferrin proteins, where glycan pattern across all of the recombinant human lactoferrin proteins can be different than a glycan pattern across all naturally occurring human lactoferrin in a human secretion (for example, breast milk).

Recombinant glycoproteins often have glycosylation profiles, patterns or fingerprint different from that found on, in or of the corresponding wild-type or naturally occurring glycoprotein. Nevertheless, the varying carbohydrates may not negatively impact glycoprotein function, such as, observed with the instant rhLF of interest, for example, binding iron. Also, carbohydrate on recombinant glycoprotein may not be as allergenic or immunogenic; or immunogenic or allergenic as wild type might be or is (Almond et al., Toxicology 301:50-57, 2012).

### B. Lactoferrin

Aspects of the present disclosure are directed to lactoferrin (LF), as well as compositions comprising lactoferrin, including edible compositions, such as, infant formula compositions. Lactoferrin (also, “lactotransferrin,”) is a whey protein found in exocrine fluids, such as, breast milk and is encoded by the LTF gene. LF binds iron (the holo form), and the apo form is free of iron. Without wishing to be bound by theory, lactoferrin is understood to have antimicrobial and anti-inflammatory properties. Aspects of the disclosure are directed to human lactoferrin (hLF) (UniProtKB/Swiss-Prot accession number P02788), including isoforms thereof. Full sequence of human lactoferrin, including signal peptide, is provided as SEQ ID NO:1. Sequence of mature human lactoferrin following cleavage of the signal peptide is provided as SEQ ID NO.2.

| | | SEQ ID |
|---|---|---|
| Protein | Sequence | NO |
| Full | MKLVFLVLLFLGALGLCLAGRRRSVQWCAVSQPEATKCFQW | 1 |
| length | QRNMRKVRGPPVSCIKRDSPIQCIQAIAENRADAVTLDGGFIYE | |
| human | AGLAPYKLRPVAAEVYGTERQPRTHYYAVAVVKKGGSFQLN | |
| lactoferrin | ELQGLKSCHTGLRRTAGWNVPIGTLRPFLNWTGPPEPIEAAVA | |
| | RFFSASCVPGADKGQFPNLCRLCAGTGENKCAFSSQEPYFSYS | |
| | GAFKCLRDGAGDVAFIRESTVFEDLSDEAERDEYELLCPDNTR | |
| | KPVDKFKDCHLARVPSHAVVARSVNGKEDAIWNLLRQAQEK | |
| | FGKDKSPKFQLFGSPSGQKDLLFKDSAIGFSRVPPRIDSGLYLG | |
| | SGYFTAIQNLRKSEEEVAARRARVVWCAVGEQELRKCNQWS | |
| | GLSEGSVTCSSASTTEDCIALVLKGEADAMSLDGGYVYTAGK | |
| | CGLVPVLAENYKSQQSSDPDPNCVDRPVEGYLAVAVVRRSDT | |
| | SLTWNSVKGKKSCHTAVDRTAGWNIPMGLLFNQTGSCKFDEY | |
| | FSQSCAPGSDPRSNLCALCIGDEQGENKCVPNSNERYYGYTGA | |
| | FRCLAENAGDVAFVKDVTVLQNTDGNNNEAWAKDLKLADFA | |
| | LLCLDGKRKPVTEARSCHLAMAPNHAVVSRMDKVERLKQVL | |
| | LHQQAKFGRNGSDCPDKFCLFQSETKNLLENDNTECLARLHG | |
| | KTTYEKYLGPQYVAGITNLKKCSTSPLLEACEFLRK | |
| Mature | GRRRSVQWCAVSQPEATKCFQWQRNMRKVRGPPVSCIKRDSP | 2 |
| human | IQCIQAIAENRADAVTLDGGFIYEAGLAPYKLRPVAAEVYGTE | |
| lactoferrin | RQPRTHYYAVAVVKKGGSFQLNELQGLKSCHTGLRRTAGWN | |
| | VPIGTLRPFLNWTGPPEPIEAAVARFFSASCVPGADKGQFPNLC | |
| | RLCAGTGENKCAFSSQEPYFSYSGAFKCLRDGAGDVAFIREST | |
| | VFEDLSDEAERDEYELLCPDNTRKPVDKFKDCHLARVPSHAV | |
| | VARSVNGKEDAIWNLLRQAQEKFGKDKSPKFQLFGSPSGQKD | |
| | LLFKDSAIGFSRVPPRIDSGLYLGSGYFTAIQNLRKSEEEVAAR | |
| | RARVVWCAVGEQELRKCNQWSGLSEGSVTCSSASTTEDCIAL | |
| | VLKGEADAMSLDGGYVYTAGKCGLVPVLAENYKSQQSSDPD | |
| | PNCVDRPVEGYLAVAVVRRSDTSLTWNSVKGKKSCHTAVDR | |
| | TAGWNIPMGLLFNQTGSCKFDEYFSQSCAPGSDPRSNLCALCI | |
| | GDEQGENKCVPNSNERYYGYTGAFRCLAENAGDVAFVKDVT | |
| | VLQNTDGNNNEAWAKDLKLADFALLCLDGKRKPVTEARSCH | |
| | LAMAPNHAVVSRMDKVERLKQVLLHQQAKFGRNGSDCPDKF | |
| | CLFQSETKNLLFNDNTECLARLHGKTTYEKYLGPQYVAGITNL | |
| | KKCSTSPLLEACEFLRK | |

In aspects, a human lactoferrin of the present disclosure is a recombinant human lactoferrin (rhlactoferrin). In aspects, a recombinant human lactoferrin of the disclosure is obtained from a mammal, fungus, yeast, bacterium or other cell. In aspects, a recombinant human lactoferrin of the disclosure is not obtained from a mammal cell. In aspects, a recombinant human lactoferrin of the disclosure is obtained from a yeast cell. The yeast cell may be, for example, from an Arxula, Aspergillus, Aurantiochytrium, Candida, Claviceps, Cryptococcus, Cunninghamella, Geotrichum, Hansenula, Kluyveromyces, Kodamaea, Komagataella, Leucosporidiella, Lipomyces, Mortierella, Ogataea, Pichia, Prototheca, Rhizopus, Rhodosporidium, Rhodotorula, Saccharomyces, Schizosaccharomyces, Trernella, Trichosporon, Wickerhamomyces or Yarrowia. In aspects, the yeast cell is a Komagataella cell (for example, Komagataella phaffii, Pichia pastoris, Komagataella pastoris, Komagataella pseudopastoris). Additional yeast species suitable for recombinant protein production are recognized in the art and contemplated herein. In aspects, a recombinant human lactoferrin of the disclosure is obtained from a bacterium. In aspects, a human lactoferrin of the disclosure is isolated from a natural source.

Aspects of the present disclosure are directed to human lactoferrin having at least one hybrid or complex N-glycan. In aspects, a human lactoferrin comprises a glycan comprising one or more of sialic acid, galactose, N-acetylgalactosamine or fucose. In aspects, a human lactoferrin comprises a bi-antennary, tri-antennary or tetra-antennary N-glycan. As disclosed herein, human lactoferrin having one or more hybrid, complex, bi-antennary, tri-antennary or tetra-antennary N-glycan may be useful in, for example, infant formula or other edible or nutritional compositions, or supplements.

Recombinant technology provides a virtually unlimited supply of a human secretory protein, amounts unattainable from natural sources because human tissue or secretions is needed and low amounts of human secretory protein in fluids and so on. Moreover, human tissue sources command rigorous testing and extraordinary purification methods to produce secure and safe, yet limiting amounts of naturally occurring secretory protein.

On the other hand, recombinant human secretory proteins of interest have at least three advantages over naturally occurring secretory protein. Because the instant method relates to a sole non-human source of secretory protein, the transformed microbe, once FDA approval of the manufacturing process of interest is obtained, that will make available unlimited amounts of recombinant secretory protein, such as, rhLF, which is less immunogenic and less allergenic than wild-type. The instant invention enables commercial development of edible compositions comprising human secretory proteins, allowing all to profit from benefits of human secretory proteins.

### C. Alpha-Lactalbumin (α-Lactalbumin)

Aspects of the present disclosure are directed to α-lactalbumin (alpha-lac, α-lac and so on), as well as compositions comprising α-lactalbumin, including an infant formula composition. α-Lactalbumin is a whey protein found in breast milk and is encoded by the LALBA gene. Aspects of the disclosure are directed to human α-lactalbumin (UniProtKB/Swiss-Prot accession number P00709), including isoforms thereof. Full sequence of human α-lactalbumin, including signal peptide, is provided as SEQ ID NO:3. Sequence of mature human α-lactalbumin following cleavage of the signal peptide is provided as SEQ ID NO:4.

| | | SEQ ID |
|---|---|---|
| Protein | Sequence | NO |
| Full length | MRFFVPLFLVGILFPAILAKQFTKCELSQLLKDIDGYGGIALPE | 3 |
| human α- | LICTMFHTSGYDTQAIVENNESTEYGLFQISNKLWCKSSQVP | |
| lactalbumin | QSRNICDISCDKFLDDDITDDIMCAKKILDIKGIDYWLAHKAL | |
| | CTEKLEQWLCEKL | |
| Mature | KQFTKCELSQLLKDIDGYGGIALPELICTMFHTSGYDTQAIVE | 4 |
| human α- | NNESTEYGLFQISNKLWCKSSQVPQSRNICDISCDKFLDDDIT | |
| lactalbumin | DDIMCAKKILDIKGIDYWLAHKALCTEKLEQWLCEKL | |

In aspects, a human α-lactalbumin of the present disclosure is a recombinant human α-lactalbumin. In aspects, a recombinant human α-lactalbumin of the disclosure is obtained from a mammal, fungus, yeast, bacterium or other cell. In aspects, a recombinant human α-lactalbumin of the disclosure is not obtained from a mammal cell. In aspects, a recombinant human α-lactalbumin of the disclosure is obtained from a yeast cell. A yeast cell may be, for example, of a genus, Arxula, Aspergillus, Aurantiochytrium, Candida, Claviceps, Cryptococcus, Cunninghamella, Geotrichum, Hansenula, Kluyveromyces, Kodamaea, Komagataella, Leucosporidiella, Lipomyces, Mortierella, Ogataea, Pichia, Prototheca, Rhizopus, Rhodosporidium, Rhodotorula, Saccharomyces, Schizosaccharomyces, Tremella, Trichosporon, Wickerhamomyces or Yarrowia. In aspects, a yeast cell is a Komagataella cell (for example, Komagataella phaffii, Pichia pastoris, Komagataella pastoris, Komagataella pseudopastoris). Additional yeasts suitable for recombinant protein production are recognized in the art and contemplated herein. In aspects, a recombinant human α-lactalbumin of the disclosure is obtained from a bacterium. In aspects, a human α-lactalbumin of the disclosure is isolated from a natural source.

Particular aspects of the present disclosure are directed to human α-lactalbumin having at least one hybrid or complex N-glycan. In aspects, a human α-lactalbumin comprises a glycan comprising one or more of sialic acid, galactose, N-acetylgalactosamine or fucose. In aspects, a human lactoferrin comprises a bi-antennary, tri-antennary or tetra-antennary N-glycan. As disclosed herein, human α-lactalbumin having one or more hybrid, complex, bi-antennary, tri-antennary, or tetra-antennary N-glycan may be useful in, for example, an infant formula or other nutritional or edible compositions, or supplements.

### D. Additional Human Milk Proteins

Additional human milk proteins contemplated in compositions (for example, edible compositions) and methods of the disclosure include, but are not limited to, as taught hereinabove for rhLF and rhaLac, and are applicable to secretory IgA (sIgA), human serum albumin, xanthine dehydrogenase, lactoperoxidase, butyrophilin, lactadherin, adiponectin, β-casein, κ-casein, leptin, osteopontin, bile salt stimulated lipase (BSSL) and lysozyme. Any one or more of those human milk proteins, the coding sequences of which are available in the art, may be included in edible compositions (for example, edible or potable compositions) of the present disclosure. Any one or more of those human milk proteins may be excluded in embodiments.

### III. EDIBLE COMPOSITION AND INFANT FORMULA

Aspects of the present disclosure are directed to edible compositions for consumption by a human, for example, an infant formula, as well as methods of use for providing nutrition to a subject, such as, an infant. The following description and that hereinabove relate also to making an edible composition of interest including nutritional compositions, such as, candy, bars, yogurt, ice cream, beverages and so on. Hence, a composition comprising lactose and a recombinant secretory protein of interest can comprise a dairy product, such as, ice cream, a milk-based nutritional beverage and so on. Any discussion herein relating to an infant formula applies also to any edible composition.

An infant formula disclosed herein may be in any form capable of consumption, or capable of dissolving, combining or diluting for consumption. In embodiments, an infant formula can be in a ready-to-consume form. In embodiments, an infant formula is in a solid concentrated form (for example, as a powder). In embodiments, an infant formula is in liquid concentrate form. An infant formula of the present disclosure may comprise one or more components useful in providing nutrition to an infant. Such components may include, but are not limited to, lactose (for example, lactose from a lactose powder), lipids (for example, lipids from one or more oils, such as, sunflower oil, safflower oil, coconut oil, canola oil, low rapeseed erucic oil etc.), protein (for example, α-lactalbumin, whey protein concentrate etc.), casein, a human milk oligosaccharide (HMO) (for example, 2′-fucosyllactose, lacto-N-neotetraose, 3-fucosyllactose, 6′-sialyllactose, difucosyllactose, lacto-N-tetraose), minerals (for example, calcium, phosphorous, iodine, iron, magnesium, copper, zinc, manganese, chloride, potassium, sodium, selenium, and salts thereof), vitamins (for example, vitamin A, vitamin D, vitamin E, vitamin K, thiamine, riboflavin, pyridoxine, niacinamide, calcium pantothenate, vitamin B6, biotin, folic acid, pantothenic acid, vitamin B12, vitamin C, choline, inositol, L-carnitine, taurine, and salts thereof), a probiotic, nucleotides (for example, cytidine 5′-monophosphate, uridine 5′-monophosphate, adenosine 5′-monophosphate, guanosine 5′-monophosphate), arachidonic acid (ARA), docosahexaenoic acid (DHA), milk fat globule membrane (MFGM) and a human milk protein (for example, secretory IgA, human serum albumin, xanthine dehydrogenase, lactoferrin, lactoperoxidase, butyrophilin, lactadherin, adiponectin, β-casein, κ-casein, leptin, lysozyme or α-lactalbumin). Any one or more, or all, of the preceding components may be included in an edible composition of the present disclosure. Any one or more of those components may be excluded from embodiments.

As described herein, an infant formula comprising a human milk protein addresses various needs by providing a formula having particular benefits previously associated with breast milk alone, including anti-inflammatory and anti-bacterial benefits. Accordingly, aspects of the present disclosure are directed to infant formula compositions comprising a human secretory protein, in cases, a human milk protein, comprising one or more human-like glycans, and methods of use thereof in providing nutrition to an infant.

In embodiments, an infant formula comprises lactose. In embodiments, an infant formula comprises at least, at most, about, or exactly 45, 46, 47, 48, 49, 50, 51, 52, 53, 54, 55, 56, 57, 58, 59, 60, 61, 62, 63, 64, 65, 66, 67, 68, 69, 70% or more lactose by weight of a dry composition, including any range or value derivable therein. In embodiments, an infant formula comprises at least 45% lactose by dry weight. In embodiments, an infant formula comprises at least 70% lactose by dry weight. An infant formula may comprise lactose from any source, including, for example, edible lactose powder. In aspects, an infant formula comprises less than 5, 4, 3, 2, 1, 0.5, 0.1, 0.01% lactose by dry weight, or less. In aspects, an infant formula does not comprise lactose.

In embodiments, an infant formula comprises one or more lipids. An infant formula may comprise one or more lipids from any source, including, for example, one or more of high oleic sunflower oil, mid-oleic sunflower oil, safflower oil, coconut oil, canola oil, low erucic rapeseed oil, sunflower oil, Mortierella alpina oil, alga oil (for example, from a Schizochytrium), soybean oil and palm oil. Any one or more, or all, of the preceding components may be included in an infant formula of the present disclosure. Any one or more of those components may be excluded from embodiments. In embodiments, an infant formula comprises at least, at most, about, or exactly 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, or 12% of any one or more of oils by weight, including any range or value derivable therein.

In embodiments, an infant formula comprises protein. An infant formula may comprise a protein from any one or more sources including, for example, whey protein lipid concentrate, whole milk powder, whey protein concentrate, non-fat dry milk, milk protein concentrate or isolate, whey protein hydrolysate, isolated protein (for example, isolated α-lactalbumin) or any other source of protein. Any one or more, or all, of the preceding components may be included in an infant formula of the present disclosure. Any one or more of those components may be excluded from embodiments. In embodiments, an infant formula comprises at least, at most, about, or exactly 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, or 12, 12.1, 12.2, 12.3, 12.4, 12.5, 12.6, 12.7, 12.8, 12.9, or 13% protein by dry weight, including any range or value derivable therein.

In embodiments, an infant formula comprises one or more human milk oligosaccharides (HMO). An HMO may be, for example, 2′-fucosyllactose or lacto-N-neotetraose. Various additional HMO's are recognized in the art and are contemplated herein. In embodiments, an infant formula comprises at least, at most, about, or exactly 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or 3% of an HMO by dry weight, including any range or value derivable therein.

In embodiments, an infant formula comprises one or more minerals. Various minerals are recognized in the art and are contemplated herein. In embodiments, an infant formula comprises one or more of calcium, phosphorous, iodine, iron, magnesium, copper, zinc, manganese, chloride, potassium, sodium, selenium, including any salt thereof. For example, an infant formula may comprise one or more (or all) of calcium phosphate, calcium citrate, magnesium chloride, potassium citrate, potassium chloride, sodium chloride, copper sulfate, ferrous sulfate, manganese sulfate, zinc sulfate, sodium selenite, calcium chloride, magnesium phosphate, sodium citrate, potassium phosphate and potassium iodide. Any one or more, or all, the preceding components may be included in an infant formula of the present disclosure. Any one or more of those components may be excluded from embodiments. In embodiments, an infant formula comprises at least, at most, about, or exactly 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or 3% of any one or more minerals by dry weight, including any range or value derivable therein. In embodiments, an infant formula comprises at least 0.1% of any one or more minerals by dry weight. In embodiments, an infant formula comprises at most, 3% of any one or more minerals by dry weight.

In embodiments, an infant formula comprises one or more vitamins. Various vitamins are recognized in the art and contemplated herein. In embodiments, an infant formula comprises one or more of vitamin A, vitamin D, vitamin E, vitamin K, thiamine, riboflavin, pyridoxine, niacinamide, calcium pantothenate, pyridoxine hydrochloride, biotin, folic acid, pantothenic acid, cyanocobalamin, vitamin C, choline, inositol, L-carnitine, inositol, and taurine, including any salt thereof. For example, in embodiments, an infant formula comprises one or more (or all) of vitamin A palmitate, vitamin D3, D-α tocopherol, vitamin K1, thiamine hydrochloride, riboflavin, niacinamide, calcium pantothenate, pyridoxine hydrochloride, biotin, folic acid, cyanocobalamin, choline bitartrate, inositol and ascorbic acid. Any one or more, or all, of the preceding components may be included in an infant formula of the present disclosure. Any one or more of those components may be excluded from embodiments. In embodiments, an infant formula comprises at least, at most, about, or exactly 0.000001, 0.00001, 0.0001, 0.001, 0.01, 0.1, or 1% of any one or more vitamins by weight, including any range or value derivable therein. In embodiments, an infant formula comprises at least 0.000001% of any one or more vitamins by weight, and at times, for liquid reagents, by volume. In embodiments, an infant formula comprises at most 1% of any one or more vitamins by weight.

In embodiments, an infant formula disclosed herein comprises one or more probiotic microbes. As used herein, a, “probiotic,” microbe as described above, comprises a microbe, such as, a bacterium or other microorganism known or suspected to have a beneficial effect on health or well-being of an individual. Various probiotics microbes are recognized in the art and are contemplated herein. Probiotic microbes which may be used in the disclosed compositions and methods include, for example, probiotic Lactobacillus and probiotic Bifidobacterium organisms. In embodiments, a probiotic microbe is Lactobacillus rhamnosus, Lactobacillus reuteri, Lactobacillus acidophilus, Lactobacillus fermentum, Lactobacillus paracasei, Lactobacillus johnsonii, Lactobacillus helveticus, Bifidobacterium breve, Bipdobacterium longum, Bifidobacterium adolescentis or Bifidobacterium infantis.

An infant formula of the disclosure may comprise at least, at most, or exactly 1, 2, 3, 4 or 5 different probiotic species of microbe. In embodiments, an infant formula does not comprise a probiotic organism. In embodiments, an infant formula comprises Bifidobacterium infantis, for example, Bifidobacterium infantis M-63. Additional beneficial probiotic microbes are disclosed in, for example, U.S. Pat. No. 9,226,521, incorporated herein by reference in entirety. An infant formula of the disclosure may comprise at least, at most, about, or exactly 10³, 10⁴, 10⁵, 10⁶, 10⁷, 10⁸, 10⁹, 10¹⁰, 10¹¹, 10¹², 10¹³, 10¹⁴, 10¹⁵ or more colony forming units (cfu), particles or other metric of probiotic microbe (for example, Bifidobacterium infantis) per 100 grams, including any range or value derivable therein.

In embodiments, an infant formula comprises one or more nucleotides. Various nucleotides, including nucleotides useful as supplements for infant formula or other food compositions, are recognized in the art and are contemplated herein. In embodiments, an infant formula comprises one or more (or all) of cytidine 5′-monophosphate, uridine 5′-monophosphate, adenosine 5′-monophosphateor guanosine 5′-monophosphate. Any one or more, or all, of the preceding components may be included in an infant formula of the present disclosure. Any one or more of those components may be excluded from embodiments.

In embodiments, an infant formula comprises one or more additional components, including, but not limited to, arachidonic acid, docosahexaenoic acid, a prebiotic, casein, such as, micellar casein, and MFGM.

In embodiments, disclosed is an infant formula comprising, in addition to one or more additional components described herein, a human secretory protein. An infant formula may comprise any human secretory protein, including human milk proteins disclosed herein, for example recombinant human milk proteins, human milk proteins comprising hybrid, complex or other human-like glycans, human whey proteins, as well as any one or more particular human milk protein, such as, lactoferrin (for example, recombinant lactoferrin). In embodiments, an infant formula comprises a naturally derived human secretory protein. In embodiments, an infant formula comprises a recombinant human secretory protein. In embodiments, an infant formula comprises a recombinant human secretory protein having a glycan pattern that is different from a glycan pattern of a corresponding natural human secretory protein.

An infant formula of the present disclosure may comprise at least, at most, about, or exactly 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1.00, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2.00% or more of one or more human secretory protein (for example, sIgA, human serum albumin, xanthine dehydrogenase, lactoferrin, lactoperoxidase, butyrophilin, lactadherin, adiponectin, β-casein, κ-casein, leptin, lysozyme, or α-lactalbumin) by dry weight, including any range or value derivable thereof.

In embodiments, an infant formula comprises about or exactly 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5% or more human lactoferrin (for example, recombinant human lactoferrin) by dry weight, or any range or value derivable therein.

In embodiments, an infant formula comprises about or exactly 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1.00, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2.00% or more dry weight of human α-lactalbumin, or any range or value derivable therein.

### IV. GENETIC ENGINEERING

Aspects of the present disclosure include recombinant protein products (for example, human secretory proteins) generated by engineered microorganisms. In aspects, disclosed are recombinant human secretory proteins (for example, recombinant lactoferrin) produced and secreted by an engineered microorganism, for example, an engineered eukaryote cell, fungus cell, yeast cell, bacterium or other cell. Vectors for transforming microorganisms in accordance with the present disclosure can be prepared by known techniques familiar to those skilled in the art in view of the disclosure herein. A vector typically contains one or more genes, in which each gene codes for expression of a desired product (a gene product) and is operably linked to one or more control sequences that regulate gene expression or target a gene product to a particular location in a recombinant cell.

Exogenous nucleic acid sequences, including, for example, nucleic acid sequences encoding fusion proteins, nucleic acid sequences encoding wild-type or mutant protein and so on, may be introduced into different host cells. Nucleic acid sequences configured to facilitate a genetic mutation in a gene also may be introduced into various host cells, as described further herein. Suitable host cells are microbial hosts that can be found broadly within fungus families. Examples of suitable host strains include but are not limited to fungus or yeast species, such as, of genera, Arxula, Aspergillus, Aurantiochytrium, Candida, Claviceps, Cryptococcus, Cunninghamella, Hansenula, Kluyveromyces, Komagataella, Leucosporidiella, Lipomyces, Mortierella, Ogataea, Pichia, Prototheca, Rhizopus, Rhodosporidium, Rhodotorula, Saccharomyces, Schizosaccharomyces, Tremella, Trichosporon and Yarrowia. Various fungus organisms are recognized in the art and contemplated herein. In embodiments, a host cell of the present disclosure is a Komagataella cell. In embodiments, a host cell of the present disclosure is Komagataella phaffii. In embodiments, a host cell of the present disclosure is Komagataella pastoris or Pichia pastoris. In embodiments, a host cell of the present disclosure is Komagataella pseudopastoris. Additional host cells include a non-fungus eukaryote cell and a bacterium.

Microbial expression systems and expression vectors are known to those skilled in the art. Any such expression vector could be used to introduce instant genes and nucleic acid sequences into an organism. Nucleic acid sequences may be introduced into appropriate microorganisms via transformation techniques. For example, a nucleic acid sequence can be cloned in a suitable plasmid, and a parent cell can be transformed with a resulting plasmid. A plasmid is not particularly limited so long as said plasmid renders a desired nucleic acid sequence inheritable to progeny of that microorganism.

Vectors or cassettes useful for transformation of suitable host cells are recognized in the art. Typically, a vector or cassette contains a gene, sequences directing transcription and translation of a relevant gene including a promoter, a selectable marker and sequences allowing autonomous replication or chromosome integration. Suitable vectors comprise a region 5′ of a gene harboring a promoter and other transcriptional initiation controls (5′ untranslated region, 5′ UTR) and a region 3′ of a DNA fragment that controls transcriptional termination (3′ UTR).

Promoters, cDNA's and 3′ UTR's, as well as other elements of vectors, can be generated through cloning techniques using fragments isolated from native sources (Green & Sambrook, “Molecular Cloning: A Laboratory Manual,” (4th ed., 2012); and U.S. Pat. No. 4,683,202; both references incorporated herein by reference in entirety). Alternatively, elements can be generated synthetically using known methods (Gene 164:49-53, 1995).

### A. Vectors and Vector Components

Vectors for transforming microorganisms in accordance with the present disclosure can be prepared by known techniques familiar to those skilled in the art in view of the disclosure herein. A vector typically contains one or more genes, in which each gene codes for expression of a desired product (the gene product) and is operably linked to one or more control sequences that regulate gene expression or target a gene product to a particular location in a recombinant cell.

### 1. Control Sequences

Control sequences are nucleic acid sequences that regulate expression of a coding sequence or direct a gene product to a particular location in or outside a cell. Control sequences that regulate expression include, for example, promoters that regulate transcription of a coding sequence and terminators that terminate transcription of a coding sequence. Another control sequence is a 3′ untranslated sequence located downstream of a coding sequence that encodes a polyadenylation signal. Control sequences that direct gene products to particular locations, for example, those that encode signal peptides, which direct a protein attached thereto to a particular location inside a cell.

Thus, an exemplary vector design for expression of a gene in a microbe contains a coding sequence for a desired gene product (for example, a selectable marker, an enzyme, a fusion protein etc.) in operable linkage with a promoter active in host yeast cells. Alternatively, if a vector does not contain a promoter in operable linkage with a coding sequence of interest, a coding sequence can be transformed into cells such that an open reading frame becomes operably linked to an endogenous promoter at vector integration. Examples of promoters contemplated herein include, but are not limited to, known and publicly available AOX1, GAP, TEF1, TPI1, DAS1, DAS2, CAT1 and FMD promoters.

A promoter used to express a gene can be a promoter naturally linked to that gene or a different promoter of a different gene.

A promoter generally can be characterized as constitutive or inducible. Constitutive promoters generally are active or function to drive expression at all times (or at certain times in cell life cycle) at the same level. Inducible promoters are active (or rendered inactive) or are significantly up-regulated or down-regulated only in response to a stimulus. Both types of promoters find application herein. Useful inducible promoters include those that mediate transcription of an operably linked gene in response to a stimulus, such as, an exogenously provided small molecule, such as, methanol, temperature (heat or cold), lack of nitrogen in culture media etc. Suitable promoters can activate transcription of an essentially silent gene or upregulate transcription of an operably linked gene that is transcribed at a low level.

Inclusion of a termination region control sequence is optional. A termination region may be native to a transcriptional initiation region (a promoter), may be native to a DNA sequence of interest or may be obtainable from another source (see, for example, Chen & Orozco, Nucleic Acids Research 16:8411, 1988).

In cases, a full nucleotide sequence of a promoter is not necessary to drive transcription, and sequences shorter than full length nucleotide sequence of a promoter can drive transcription of an operably-linked gene. A minimal portion of a promoter, termed a core promoter, include a transcription start site, a binding site for a RNA polymerase and a binding site for a transcription factor.

A promoter may be linked to a target by introducing a promoter and a target into a nucleic acid molecule, for example, a vector. A vector may be introduced into a cell, thereby expressing promoter and target. In an embodiment, a promoter is linked to a target by introducing a promoter into DNA of a cell, for example, via homologous recombination, thereby integrating a promoter into a genome of a cell.

### B. Genes and Codon Optimization

Typically, a gene includes a promoter, a coding sequence and termination control sequence. When assembled by recombinant DNA technology, a gene may be termed an expression cassette and may be flanked by restriction sites for convenient insertion into a vector that is used to introduce that recombinant gene into a host cell. An expression cassette can be flanked by DNA sequences from a genome or other nucleic acid target to facilitate stable integration of an expression cassette into a genome by homologous recombination. Alternatively, a vector and expression cassette may remain unintegrated (for example, an episome), in which case, a vector typically includes an origin of replication, which is capable of providing for replication of a vector DNA.

A common gene present on a vector is a gene encoding a protein, expression of which allows a recombinant cell containing a protein to be differentiated from cells that do not express that protein. Such a gene, and corresponding gene product, is called a selectable marker or selection marker. Any of a wide variety of selectable markers can be employed in a transgene (foreign gene, recombinant gene or gene of interest) construct useful for transforming organisms covered in the disclosed embodiments.

For optimal expression of a recombinant protein, it may be beneficial to employ coding sequences that produce mRNA with codons optimally used by a host cell to be transformed. Thus, proper or maximal expression of transgenes can require codon usage of a transgene to match specific codon bias of an organism in which a transgene is being expressed. Precise mechanisms underlying such effect are many, but include proper balancing of available aminoacylated tRNA pools with proteins being synthesized in a cell, coupled with more efficient translation of transgene messenger RNA (mRNA) when the need is met. When codon usage in a transgene is not optimized, available tRNA pools may not be sufficient to allow for efficient translation of a transgene mRNA resulting in ribosomal stalling and termination, and possible instability of a transgene mRNA.

A coding sequence of the present disclosure can be codon optimized for a particular host cell by replacing one or more rare codons with one or more codons more frequently found in a host cell. A rare codon in a host cell is a codon found in less than 5%, less than 10% or less than 20% of coding sequences in a host cell. Rare codons can be identified using methods known to those of skill in the art.

Aspects of the disclosure comprise transformation of a microorganism with a nucleic acid sequence comprising a gene that encodes a protein. A gene may be native to a cell or from a different species. A gene may be derived from a different species yet modified (for example, codon optimized) for optimal expression in a microorganism. In embodiments, a gene is inheritable in progeny of a transformed cell. In embodiments, a gene is inheritable by residing on a plasmid. In embodiments, a gene is inheritable by integrating into a genome of a transformed cell.

Further aspects of the disclosure may comprise transformation of a microorganism with a nucleic acid sequence configured to generate a mutation in a gene of a microorganism. For example, aspects of the disclosure may comprise transformation of a microorganism with a nucleic acid sequence comprising sequences upstream and downstream of a gene (for example, an OCH1 gene encoding a 1,6-mannosyl transferase of yeast), thereby facilitating reduced expression or deletion of a gene via homologous recombination. Various methods for generating mutations (including deletions or knockout mutations, as well as mutations which reduce expression of a gene) in genes of a microorganism are recognized in the art and envisioned herein. A microorganism having a deletion or knockout mutation of a gene does not produce a functional copy of a protein. For example, a recombinant yeast cell of the disclosure may comprise a deletion of an endogenous OCH1 gene, such that a recombinant yeast cell does not express an endogenous, functional OCH1 protein. A microorganism having reduced expression of a gene or protein can produce a functional copy of a protein, but at a reduced level as compared to wild-type (that is, a non-recombinant or non-genetically modified) microorganism of the same species. Methods for reducing expression of a protein are recognized in the art and include, for example, replacement of an endogenous promoter and/or modification of one or more regulatory elements.

### C. Transformation

Cells can be transformed by any suitable technique including, for example, use of biolistic devices, electroporation, glass bead transformation, silicon carbide whisker transformation and so on. Any convenient technique for introducing a transgene into a microorganism can be employed in embodiments disclosed herein.

Vectors for transformation of microorganisms can be prepared by known techniques familiar to those skilled in the art. In an embodiment, an exemplary vector design for expression of a gene in a microorganism contains a gene encoding an enzyme in operable linkage with a promoter active in a microorganism. Alternatively, if a vector does not contain a promoter in operable linkage with a gene of interest, a gene can be transformed into cells to be operably linked to a native promoter at vector integration. A vector also can contain a second gene that encodes a protein. Optionally, one or both gene(s) is/are followed by a 3′ untranslated sequence containing a polyadenylation signal. Expression cassettes encoding those two genes can be physically linked in a vector or reside on separate vectors. Co-transformation of microbes also can be used, in which distinct vector molecules are simultaneously used to transform cells (Protist 155:381-93, 2004). Transformed cells optionally can be selected based on ability to grow in presence of an antibiotic or other selectable marker under conditions in which cells lacking a resistance cassette would not grow or flourish.

### D. Genetically Engineered Cells

Aspects of the disclosure comprise genetically engineered cells (also, “engineered cells,” and, “recombinant cells,”) and methods for making and using such cells. In embodiments, disclosed do recombinant cells comprise one or more exogenous nucleic acid sequences. Also disclosed are methods for generating such recombinant cells comprising introducing one or more exogenous nucleic acid sequences into a host cell. Further described are methods for collecting one or more products (for example, a mammal glycoprotein) from such recombinant cells comprising culturing cells and collecting product.

In embodiments, a recombinant cell is a prokaryote cell, such as, a bacterium. In embodiments, a recombinant cell is a eukaryote cell, such as, a mammal cell, a yeast cell, a filamentous fungus cell, a protist cell, an alga cell, a bird cell, a plant cell, an insect cell and so on. In embodiments, a recombinant cell is not a mammal cell. In embodiments, a host cell is a yeast cell. Those with skill in the art will recognize that many forms of filamentous fungus produce yeast-like growth, and definition of yeast herein encompasses such cells. A recombinant cell of the disclosure may be selected from the group consisting of alga, bacterium, mold, fungus, plant and yeast. In embodiments, a recombinant cell of the disclosure is a bacterium (for example, E. coli), a fungus cell or a yeast cell, such as, Saccharomyces cerevisiae.

In embodiments, a recombinant cell of the disclosure is a recombinant yeast cell. A recombinant yeast cell may be any suitable yeast cell recognized in the art. In aspects, the yeast cell is an Arxula, Aspergillus, Aurantiochytrium, Candida, Claviceps, Cryptococcus, Cunninghamella, Geotrichum, Hansenula, Kluyveromyces, Kodamaea, Komagataella, Leucosporidiella, Lipomyces, Mortierella, Ogataea, Pichia, Prototheca, Rhizopus, Rhodosporidium, Rhodotorula, Saccharomyces, Schizosaccharomyces, Tremella, Trichosporon, Wickerhamomyces or Yarrowia cell. In embodiments, a yeast cell is Arxula adeninivorans, Aspergillus niger, Aspergillus orzyae, Aspergillus terreus, Aurantiochytrium limacinum, Candida utilis, Claviceps purpurea, Cryptococcus albidus, Cryptococcus curvatus, Cryptococcus ramirezgomezianus, Cryptococcus terreus, Cryptococcus wieringae, Cunninghamella echinulata, Cunninghamella japonica, Geotrichum fermentans, Hansenula polymorpha, Kluyveromyces lactis, Komagataella phaffii, Komagataella pastoris, Komagataella pseudopastoris, Kluyveromyces marxianus, Kodamaea ohmeri, Leucosporidiella creatinivora, Lipomyces lipofer, Lipomyces starkeyi, Lipomyces tetrasporus, Mortierella isabellina, Mortierella alpina, Ogataea polymorpha, Pichia ciferrii, Pichia guilliermondii, Pichia pastoris, Pichia stipites, Prototheca zopfii, Rhizopus arrhizus, Rhodosporidium babjevae, Rhodosporidium toruloides, Rhodosporidium paludigenum, Rhodotorula glutinis, Rhodotorula mucilaginosa, Saccharomyces cerevisiae, Schizosaccharomyces pombe, Tremella enchepala, Trichosporon cutaneum, Trichosporon fermentans, Wickerhamomyces ciferrii or Yarrowia lipolytica.

In embodiments, a yeast cell is a Komagataella cell. In embodiments, a yeast cell is Kluyveromyces phaffii, Pichia pastoris, Komagataella pastoris or Komagataella pseudopastoris. In embodiments, a yeast cell is Kluyveromyces phaffii cell.

### V. MANUFACTURING

Edible compositions according to the present invention may be prepared by any known or otherwise suitable manner. For example, an infant formula may be obtained by blending a protein with a human secretory protein, and optionally, a carbohydrate source and a lipid source, in appropriate proportions. Emulsifiers may be included. Vitamins and minerals may be added, but also may be added later during manufacture to avoid thermal degradation. Water, such as, water subjected to purification, such as, reverse osmosis or deionized water, then may be added and mixed to form a liquid mixture. Temperature of mixing can be room temperature, but also may be higher to facilitate suspension and mixing, though below temperatures that might damage an ingredient.

If desired, a liquid mixture is dried in a suitable drying apparatus, such as, a spray drier or freeze drier, and can be converted into or to a powder.

Powdered infant formula may be produced using various processes, such as, dry blending dehydrated ingredients to constitute a uniform mixture or hydrating and wet-mixing a mixture of ingredients, such as, fat, protein and carbohydrate ingredients and then evaporating or spray drying the resultant mixture. A combination of processes may be used where a base powder is first produced by wet-mixing and spray drying all or some of the macro-ingredients and then dry blending the remaining ingredients, including carbohydrate, minerals and vitamins and other micronutrients, to create a final composition. Liquid formulae are available in a ready-to-feed format or as a concentrated liquid, which requires dilution, such as, 1:1, with water.

To produce a liquid infant formula, a homogenized mixture is filled into suitable containers, preferably aseptically. However, a liquid composition may be retorted in a suitable container, using suitable apparatus for filling and retorting using commercially available machinery.

As mentioned above, the invention relates to ready-to-drink formulations and processes for preparing ready-to-drink formulations. A liquid food may be a beverage, such as, a nutraceutical drink, an energy drink, a milkshake, a performance or nutrition product, such as, a protein shake, and so on.

Steps that eliminate or restrict microbial growth are practiced during production. In general, for example, preservation of an oil-in-water (o/w) emulsion by dehydration for powder products or sterilization in the case of ready-to-feed or concentrated liquid products. A liquid mixture then may be thermally treated or sterilized by another method to reduce risk of spoilage. A mixture then may be homogenized.

A manufacture of the invention can involve high-temperature-processing at about 110 °C or above.

During high-temperature-processing (UHT), an edible composition is heated sufficiently to sterilize a liquid composition, which may lengthen shelf-life of a composition or formulation.

High-temperature-processing may be carried out at about 110 °C to about 150 °C. For example, high-temperature-processing may be carried out at about 110 °C to about 145 °C, about 110 °C to about 140 °C, about 110 °C to about 135 °C, about 110 °C to about 130 °C, about 110 °C to about 125 °C, about 110 °C to about 120 °C, about 110 °C to about 115 °C, about 115 °C to about 150 °C, about 115 °C to about 145 °C, about 115 °C to about 140 °C, about 115 °C to about 135 °C, about 115 °C to about 130 °C, about 115 °C to about 125 °C, about 115 °C to about 120 °C, about 120 °C to about 150 °C, about 120 °C to about 145 °C, about 120 °C to about 140 °C, about 120 °C to about 135 °C, about 120 °C to about 130 °C, about 120 °C to about 125 °C, about 125 °C to about 150 °C, about 125 °C to about 145 °C, about 125 °C to about 140 °C, about 125 °C to about 135 °C, about 125 °C to about 130 °C, about 130 °C to about 150 °C, about 130 °C to about 145 °C, about 130 °C to about 140 °C, about 130 °C to about 135 °C, about 135 °C to about 150 °C, about 135 °C to about 145 °C, about 135 °C to about 140 °C, about 140 °C to about 150 °C, about 140 °C to about 145 °C, or about 145 °C to about 150 °C.

High-temperature-processing may be carried out for about 5 seconds to about 60 minutes, for about 5 seconds to about 45 minutes, about 5 seconds to about 30 minutes, about 5 seconds to about 15 minutes, about 5 seconds to about 5 minutes, about 5 seconds to about 120 seconds, about 5 seconds to about 60 seconds, about 5 seconds to about 30 seconds, about 30 seconds to about 60 minutes, about 30 seconds to 45 minutes, about 30 seconds to about 30 minutes, about 30 seconds to about 15 minutes, about 30 seconds to about 5 minutes, about 30 seconds to about 120 seconds, about 30 seconds to about 60 seconds, about 60 seconds to about minutes, about 60 seconds to about 45 minutes, about 60 seconds to about 30 minutes, about 60 seconds to about 15 minutes, about 60 seconds to about 5 minutes, about 60 seconds to about 120 seconds, about 120 seconds to about 60 minutes, about 120 seconds to about 45 minutes, about 120 seconds to about 30 minutes, about 120 seconds to about 15 minutes, about 120 seconds to about 5 minutes, about 5 minutes to about 60 minutes, about 5 minutes to about 45 minutes, about 5 minutes to about 30 minutes, about 5 minutes to about 15 minutes, about 15 minutes to about 60 minutes, about 15 minutes to about 45 minutes, about 15 minutes to about 30 minutes, about 30 minutes to about 60 minutes, about 30 minutes to about 45 minutes, or about 45 minutes to about 60 minutes.

High-temperature-processing may be carried out by direct or indirect heating. For example, high-temperature-processing may be carried out by steam injection, heat exchange or using a retort.

An edible composition can be treated with high pressure prior to packaging and sale, with high pressure enhancing safety and prolonging shelf life. High pressure processing (HPP) is known and requires pressures of about 6000 bar or more. Pressure of 50 MPa to about 1000 MPa can be used at room temperature or lower, see, for example, U.S. Pat. Nos. 5,213,029, 5,316,745; 5,683,735; and 6,033,717; and US Pub No 20080050507.

An edible composition of interest is sterile filled into a suitable packaging, such as, a lined can, a pouch, a package wrapping, a powder container, a bottle and so on, which is sealed, optionally treated for quality control and shelf stability, and distributed for sale.

All references cited herein, are incorporated herein by reference in entirety.

The instant disclosure now will be exemplified in the following non-limiting examples. It should be understood that the Examples, while indicating embodiments of the invention, are given by way of illustration only. From the above discussion and the Examples, one skilled in the art can ascertain essential characteristics of the instant invention, and without departing from the spirit and scope thereof, can make various changes and modifications of the invention to adapt same to various uses and conditions.

Herein, percentages of dry ingredients are on a weight basis. For liquid compositions, percentages can be on a weight basis as water generally is the predominant medium with a density of 1 g/cc, or on a weight per volume basis. Percentages of liquid ingredients in a dry formulation can be on a weight basis. Ratios of ingredients in a dry formulation can be used to determine percentages of reagents in an analogous liquid formulation based on the targeted solids level in the analogous liquid formulation.

### EXAMPLES

### Example 1

A batch of a milk-based infant formula was prepared according to the formulation in the table below. Lactose was hydrated at 45-55% solids in 150-160 °F water, then proteins were added. The vegetable oil blend, ARA-DHA blend and fat soluble vitamin premix were mixed into lactose. Mineral and trace minerals, as well as other minerals, vitamins and nutrients were added. The mix was held at 140-145 °F with high agitation during mixing, then the mixture was homogenized at 2000-3500 PSI and cooled at 40 °F. Nucleotide premix was added to the cold mix and agitated before pasteurization at 170-180 °F for 60 seconds followed by spray-drying to a resulting moisture content of 1.5-3.5%. Probiotic microorganisms and human lactoferrin were added to the base formulation via dry-blending.

The powder was mixed with water at a rate of 1 tablespoon to 2 ounces of water to yield a drinkable infant formula.

TABLE 1

| Ingredient | Item Code | % Range |
|---|---|---|
| Lactose | — | 45-49% |
| Vegetable Oil blend | — | 20-22% |
| Whole Milk Powder | — | 14-17% |
| Whey Protein Concentrate 80% | — | 4.0-7.0% |
| Alpha-Lac enriched Whey Protein | — | 1.5-4.0% |
| Mineral Premix | XR73983000 | 1.0-3.5% |
| 2′-Fucosyllactose (HMO) | — | 1.5-2.0% |
| ARA-DHA Blend | — | 0.5-1.0% |
| Choline Bitartrate | — | 0.2-0.5% |
| Vitamin Premix | XR74360000 | 0.2-0.6% |
| Potassium Hydroxide | — | 0.1-0.3% |
| Ascorbic Acid | — | 0.1-0.3% |
| Potassium Bicarbonate | — | 0.05-0.12% |
| Trace Mineral Premix | XR73965000 | 0.02-0.07% |
| Nucleotide Premix | XR73202000 | 0.01-0.05% |
| Liquid Fat Soluble Premix | XR74558000 | 0.01-0.05% |
| Human Lactoferrin | — | 0.4-1.0% |
| Probiotic Microbes | — | 0.05-0.2% |
| Moisture | — | 1.5-3.5% |

### Example 2

A batch of milk-based infant formula containing lower amount of lactose is prepared as provided in Example 1 using the formulation provided in the table below.

TABLE 2

| Ingredient | Item Code | % Range |
|---|---|---|
| Corn Syrup Solids | — | 50-55% |
| Vegetable Oil blend | — | 22-27% |
| Milk Protein Isolate | — | 4.0-7.0% |
| Whey Protein Concentrate 80% | — | 4.0-7.0% |
| Alpha-Lac enriched Whey Protein | — | 1.5-4.0% |
| Mineral Premix | XR73983000 | 1.0-3.5% |
| 2′-Fucosyllactose (HMO) | — | 1.5-2.0% |
| ARA-DHA Blend | — | 0.5-1.0% |
| Choline Bitartrate | — | 0.2-0.5% |
| Vitamin Premix | XR74360000 | 0.2-0.6% |
| Potassium Hydroxide | — | 0.1-0.3% |
| Ascorbic Acid | — | 0.1-0.3% |
| Potassium Bicarbonate | — | 0.05-0.12% |
| Trace Mineral Premix | XR73965000 | 0.02-0.07% |
| Nucleotide Premix | XR73202000 | 0.01-0.05% |
| Liquid Fat Soluble Premix | XR74558000 | 0.01-0.05% |
| Human Lactoferrin | — | 0.4-1.0% |
| Probiotic Microbes | — | 0.05-0.2% |
| Moisture | — | 1.5-3.5% |

### Example 3

A batch of soy-based infant formula is prepared as provided in Example 1 and using the formulation provided in the table below.

TABLE 3

| Ingredient | Item Code | % Range |
|---|---|---|
| Corn Syrup Solids | — | 52-57% |
| Vegetable Oil blend | — | 22-27% |
| Soy Protein Isolate | — | 11-14% |
| Mineral Premix | XR73983000 | 1.0-3.5% |
| 2′-Fucosyllactose (HMO) | — | 1.5-2.0% |
| ARA-DHA Blend | — | 0.5-1.0% |
| Choline Bitartrate | — | 0.2-0.5% |
| Vitamin Premix | XR74360000 | 0.2-0.6% |
| Potassium Hydroxide | — | 0.1-0.3% |
| Ascorbic Acid | — | 0.1-0.3% |
| Potassium Bicarbonate | — | 0.05-0.12% |
| Trace Mineral Premix | XR73965000 | 0.02-0.07% |
| Liquid Fat Soluble Premix | XR74558000 | 0.01-0.05% |
| Human Lactoferrin | — | 0.4-1.0% |
| Probiotic Microbes | — | 0.05-0.2% |
| Moisture | — | 1.5-3.5% |

### Example 4

A batch of milk-based infant formula containing partially hydrolyzed whey is prepared as provided in Example 1 using the formulation noted in the table below.

TABLE 4

| Ingredient | Item Code | % Range |
|---|---|---|
| Lactose | — | 25-30% |
| Maltodextrin | — | 23-27% |
| Vegetable Oil blend | — | 23-27% |
| Whey Protein Hydrolysate | — | 10-12% |
| Alpha-Lac enriched Whey Protein | — | 1.5-4.0% |
| Mineral Premix | XR73983000 | 1.0-3.5% |
| 2′-Fucosyllactose (HMO) | — | 1.5-2.0% |
| ARA-DHA Blend | — | 0.5-1.0% |
| Choline Bitartrate | — | 0.2-0.5% |
| Vitamin Premix | XR74360000 | 0.2-0.6% |
| Potassium Hydroxide | — | 0.1-0.3% |
| Ascorbic Acid | — | 0.1-0.3% |
| Potassium Bicarbonate | — | 0.05-0.12% |
| Trace Mineral Premix | XR73965000 | 0.02-0.07% |
| Nucleotide Premix | XR73202000 | 0.01-0.05% |
| Liquid Fat Soluble Premix | XR74558000 | 0.01-0.05% |
| Human Lactoferrin | — | 0.4-1.0% |
| Probiotic Organisms | — | 0.05-0.2% |
| Moisture | — | 1.5-3.5% |

### Example 5

A batch of milk-based infant formula ready-to-feed is prepared as follows, using the formulation provided in Table 5.

A quantity of reverse osmosis (RO) water representing 50% of the total quantity is heated to 150-160 °F. Proteins and carrageenan are hydrated using high shear agitation. While maintaining high agitation, the vegetable oil blend, ARA-DHA blend and fat soluble vitamin premix are added followed by the lactose. Mineral and trace minerals as well as other minerals, vitamins and nutrients are added. The mix is held at 140-145 °F at high agitation during mixing, then pasteurized at 185-190 °F for 30 seconds, homogenized at 2000-3500 PSI and cooled at 40 °F. Nucleotide premix and human lactoferrin are added to the cold mix along with the residual RO water to bring the total solids to 12.8-13.2% w/w. The mix is UHT-treated and filled aseptically in 8 oz plastic bottles.

TABLE 5

| Ingredient | Item Code | % Range |
|---|---|---|
| Reverse Osmosis Water | — | 86-90% |
| Lactose | — | 6-8% |
| Vegetable Oil blend | — | 2-4% |
| Whole Milk Powder | — | 1-3% |
| Whey Protein Concentrate 80% | — | 0.5-0.9% |
| Alpha-Lac enriched Whey Protein | — | 0.3-0.5% |
| Mineral Premix | XR73983000 | 0.2-0.4% |
| 2′-Fucosyllactose (HMO) | — | 0.2-0.3% |
| Human Lactoferrin | — | 0.003-0.006% |
| ARA-DHA Blend | — | 0.05-0.12% |
| Choline Bitartrate | — | 0.02-0.06% |
| Vitamin Premix | XR74360000 | 0.02-0.04% |
| Potassium Hydroxide | — | 0.01-0.03% |
| Ascorbic Acid | — | 0.01-0.03% |
| Potassium Bicarbonate | — | 0.01-0.02% |
| Carrageenan | — | 0.005-0.007% |
| Trace Mineral Premix | XR73965000 | 0.005-0.007% |
| Nucleotide Premix | XR73202000 | 0.003-0.006% |
| Liquid Fat Soluble Premix | XR74558000 | 0.003-0.006% |

### Example 6

A batch of ready to use liquid nutritional beverage is prepared according to the formulation in Table 6. A quantity of reverse osmosis (RO) water representing 50% of the total quantity is heated to 150-160 °F. Proteins (milk protein concentrate and soy protein isolate), cellulose gel & gum and carrageenan are hydrated using high shear agitation. While maintaining high agitation, the oils (high oleic safflower oil and canola oil), lecithin and fat soluble vitamin premix are added, followed by the maltodextrin, sugar and fructooligosaccharides. Individual minerals, the trace mineral premix, the water soluble vitamin premix and other nutrients are added. The mix is held at 140-145 °F at high agitation during mixing, then the mixture is pasteurized at 185-190 °F for 30 seconds, homogenized at 2000-3500 PSI and then is cooled at 40 °F. Vitamin C in 5% solution with a portion of potassium hydroxide and human lactoferrin in 10% solution along with flavors are added to the cold mix along with the residual RO water to bring the total solids to 22.8-24.2% w/w The mix is UHT-treated and filled aseptically in 8 oz plastic bottles.

TABLE 6

| Ingredients | % Range |
|---|---|
| Water | 76-78 |
| Maltodextrin | 8.0-10 |
| Sugar | 4.0-5.0 |
| Milk Protein Concentrate | 2.5-3.0 |
| High Oleic Safflower oil | 2.2-2.9 |
| Soy Protein Isolate | 1.8-2.5 |
| Canola oil | 0.8-1.2 |
| Fructooligosaccharides | 0.4-0.6 |
| Tricalcium Phosphate | 0.2-0.4 |
| Magnesium Chloride | 0.2-0.3 |
| Lecithin | 0.1-0.2 |
| Sodium Citrate | 0.1-0.2 |
| Potassium Citrate | 0.08-0.15 |
| Sodium Hydroxide | 0.08-0.13 |
| Magnesium Phosphate | 0.08-0.13 |
| Potassium hydroxide | 0.08-0.12 |
| Vitamin C | 0.04-0.06 |
| Human lactoferrin | 0.04-0.06 |
| Potassium Chloride | 0.04-0.06 |
| Natural & artificial flavors | 0.04-0.06 |
| Choline Chloride | 0.04-0.06 |
| Trace mineral premix | 0.03-0.04 |
| Potassium Phosphate | 0.03-0.04 |
| Water soluble vitamin premix | 0.015-0.021 |
| Cellulose gel and gum | 0.010-0.015 |
| Fat soluble vitamin premix | 0.005-0.010 |
| Carrageenan | 0.001-0.003 |
| Potassium Iodide | 0.00001-0.00003 |

### Example 7

A batch of ready to use liquid nutritional low glycemic index beverage is prepared as provided in Example 6 using the formulation of Table 7.

TABLE 7

| Ingredients | % Range |
|---|---|
| Water | 76-77 |
| Maltodextrin | 5.8-6.5 |
| Acid Casein | 4.0-4.5 |
| Fructose | 2.5-3.0 |
| High Oleic Safflower oil | 2.5-3.0 |
| Maltitol | 2.5-3.0 |
| Resistant starch | 0.7-0.9 |
| Caseinate | 0.5-0.7 |
| Fructooligosaccharides | 0.4-0.6 |
| Soy fiber | 0.4-0.5 |
| Canola oil | 0.2-0.4 |
| Tricalcium Phosphate | 0.2-0.4 |
| Magnesium Chloride | 0.2-0.3 |
| Lecithin | 0.1-0.2 |
| Sodium Citrate | 0.1-0.2 |
| Potassium Citrate | 0.08-0.15 |
| Sodium Hydroxide | 0.08-0.13 |
| Magnesium Phosphate | 0.08-0.13 |
| m-inositol | 0.08-0.12 |
| Potassium hydroxide | 0.08-0.12 |
| Vitamin C | 0.04-0.06 |
| Human lactoferrin | 0.04-0.06 |
| Potassium Chloride | 0.04-0.06 |
| Choline Chloride | 0.04-0.06 |
| Trace mineral premix | 0.03-0.04 |
| Potassium Phosphate | 0.03-0.04 |
| Carnitine | 0.02-0.03 |
| Water soluble vitamin premix | 0.015-0.021 |
| Gellan gum | 0.010-0.015 |
| Taurine | 0.005-0.015 |
| Fat soluble vitamin premix | 0.005-0.010 |
| Lutein (20% in safflower oil) | 0.001-0.003 |
| Potassium Iodide | 0.00001-0.00003 |

### Example 8

A batch of nutritional beverage powder is prepared using the formulation of Table 8. Proteins (milk protein concentrate and soy protein isolate) are hydrated at 45-55% solids in 145-150 °F water, then the oils (high oleic safflower oil and canola oil), lecithin and fat soluble vitamin premix are added followed by the maltodextrin, sugar and fructooligosaccharides. Mineral and trace minerals as well vitamins and nutrients are added. The mix is held at 140-145 °F at high agitation during mixing, then homogenized at 2000-3500 PSI and cooled at 40 °F. Vitamin C in 5% solution with a portion of potassium hydroxide and human lactoferrin in 10% solution along with N&A flavors are added to the cold mix. The mix is pasteurized at 170-180 °F for 60 seconds followed by spray-drying to a resulting moisture content of 2.5-4.0%.

Optionally, the human lactoferrin is added to the base formulation via dry-blending.

TABLE 8

| Ingredients | % Range |
|---|---|
| Maltodextrin | 35-40 |
| Sugar | 16-20 |
| Milk Protein Concentrate | 10- 13 |
| High Oleic Safflower oil | 10-13 |
| Soy Protein Isolate | 8-10 |
| Canola oil | 4-5 |
| Fructooligosaccharides | 1.8-2.2 |
| Tricalcium Phosphate | 1.1-1.3 |
| Magnesium Chloride | 0.9-1.1 |
| Lecithin | 0.8-1.0 |
| Sodium Citrate | 0.4-0.6 |
| Potassium Citrate | 0.4-0.6 |
| Sodium Hydroxide | 0.4-0.6 |
| Magnesium Phosphate | 0.4-0.5 |
| Potassium hydroxide | 0.08-0.12 |
| Vitamin C | 0.04-0.06 |
| Human lactoferrin | 0.04-0.06 |
| Potassium Chloride | 0.04-0.06 |
| Natural & artificial flavors | 0.18-0.25 |
| Choline Chloride | 0.18-0.25 |
| Trace mineral premix | 0.15-0.18 |
| Potassium Phosphate | 0.14-0.17 |
| Water soluble vitamin premix | 0.05-0.09 |
| Fat soluble vitamin premix | 0.02-0.04 |
| Potassium Iodide | 0.00007-0.00009 |
| Moisture | 2.5-4.0 |

### Example 9

Stable juice-based protein beverages are prepared by mixing a fruit juice with 0.1-10.0% of protein (for example, casein, lactalbumin, serum albumin, glycomacropeptide, soy protein, rice protein, pea protein, whey protein, canola protein, wheat protein, hemp protein, zein, flax protein, egg white protein, ovalbumin, gelatin protein and combinations thereof) using the formulation of Table 9. pH of the admixture is adjusted to between 2 and 3.4. Optionally, one or more of an anti-foaming agent, a nutrient, calcium, an herbal supplement, a flavoring agent, a sweetener, a coloring agent, a preservative and an energy-generating additive selected from caffeine, magnesium and citrulline malate) are added to the mixture. Microbes are inactivated by high pressure processing (HPP). The protein beverage is packaged in a container which may be stored without refrigeration for more than one year before use by a consumer of the protein beverage.

TABLE 9

| Ingredient | % Range |
|---|---|
| Fruit Juice Blend | 80-98% |
| Protein | 0.1-10% |
| Prebiotic | 0.1-5.0% |
| Human Lactoferrin | 0.1-3.0% |
| Zinc Citrate | 0.1-0.3% |
| Vitamin D | 0.1-0.3% |

### Example 10

A carbonated milk beverage with enhanced shelf stability is prepared based on the formulation provided in Table 10. First, skim milk powder solution is heated at least 85-138 °C for 5 seconds. The milk liquid dairy product is ultra-heat treated at least at 150 °C and at a pressure of 700 kPa. In the blending tank, other ingredients are added and homogenized at 2,000-5,000 psi for 5 min. The liquid dairy product is cooled to a temperature of less than 10 °C. The cooled liquid dairy product is subject to pressurized carbon dioxide so that at least 3-8 vols. of carbon dioxide are dissolved in the liquid dairy product. The carbonated liquid dairy product is packaged in shelf-stable closed containers that do not require refrigeration.

TABLE 10

| Ingredient | % Range |
|---|---|
| Water | 80-90% |
| Corn Syrup | 5-10% |
| Sugar | 2-8% |
| Liquefied Carbon Dioxide | 0.5-1.5% |
| Skim Milk Powder | 0.5-1.5% |
| Human Lactoferrin | 0.5-1.5% |
| Flavor | 0.1-1.0% |
| Citric Acid | 0.05-0.2% |
| Phosphoric Acid | 0.01-0.05% |
| Natural Food Color | 0.01-0.05% |
| Vitamin C | 0.005-0.05% |
| Vitamin A | 0.005-0.05% |
| Vitamin D | 0.005-0.05% |

### Example 11

A protein-fortified frozen dessert formulation is prepared using the formulation described in Table 11. The ingredients are blended at 5,000-7,000 rpm to prepare a sweet mix. The sweet mix is preheated at 30-60 °C. The preheated sweet mix is pasteurized by heating at 69 °C for 30 minutes (batch) or at 80 °C for 25 seconds (continuous). The sweet mix is homogenized at 2000-3000 psi (one-stage homogenization) or at 2000-2500 psi, followed by 500 psi (two-stage homogenization). Homogenized sweet mix is cooled to 3-5 °C for 4-6 hours to produce an aged sweet mix and the aged mix is frozen to prepare a protein-fortified frozen dessert.

TABLE 11

| Ingredient | % Range |
|---|---|
| Water | 50-65% |
| Sugar | 10-20% |
| Milk Solids | 10-15% |
| Fat | 0.1-20% |
| Flavoring Agent | 0.1-10% |
| Protein Component | 0.1-11% |
| Human Lactoferrin | 0.1-2.0% |

### Example 12

A protein-fortified spreadable yogurt product is prepared according to the formulation provided in Table 12 by combining a casein-containing ingredient with milk to make yogurt milk. Yogurt culture is added to the yogurt milk and allowed to ferment. The resulting yogurt mixture is separated into a yogurt retentate and a yogurt permeate. Yogurt retentate is used as a spreadable protein-fortified yogurt. The spreadable yogurt can be flavored and then packaged.

TABLE 12

| Ingredient | % Range |
|---|---|
| Milk | 85-95% |
| Casein-Containing Ingredient | 5-15% |
| Human Lactoferrin | 0.1-2.0% |
| Yogurt Culture | 0.1-1.0% |
| Flavor Agent | 0-1.0% |

### Example 13

A gummy snack with high protein is produced using the formulation in the following table. A mixture is formed by mixing water, glucose syrup, first sugar alcohol and second sugar alcohol. The mixture is cooked at 230-285 °F for 10-60 minutes to form a sugar mixture, which then is cooled to 60-80 °F. An acid solution (malic acid) is added to form a sugar-acid mixture of pH 2-4. The sugar-acid mixture is mixed with a gelatin for 4-12 minutes to form a sugar-gelatin mixture. The sugar-gelatin mixture is mixed with protein to form a gel mixture. The gel mixture then is processed by machinery for forming shapes.

TABLE 13

| Ingredient | % Range |
|---|---|
| Whey Protein Concentrate | 10-50% |
| Water | 5-35% |
| Sugar Alcohol 1 | 5-30% |
| Sugar Alcohol 2 | 5-20% |
| Gelatin | 0.1-15% |
| Glucose Syrup | 0.1-15% |
| Human Lactoferrin | 0.1-3.0% |

### Example 14

A high protein snack food is produced using the formulation provided in Table 14. Any of a variety of protein particles (for example, chicken powder, pea protein powder etc.) are extruded to form protein pieces. The pieces or crisp and the remaining ingredients are combined and the mixture is used to form a bar, which is treated at 160 °F until an internal temperature of 140 °F is reached.

TABLE 14

| Ingredient | % Range |
|---|---|
| Extruded protein crisp | 15-35% |
| High Maltose Corn Syrup | 15-25% |
| Almonds Raw | 10-15% |
| Dried Blueberries | 10-15% |
| Pumpkin Seeds | 5-15% |
| Sunflower Butter | 5-10% |
| Inverted Sugar | 3-8% |
| Canola Oil | 0.1-3.0% |
| Human Lactoferrin | 0.1-3.0% |
| Glycerin | 0.1-2.0% |
| Lecithin | 0.1-1.0% |

### Example 15

A batch of nutritional plant-based beverage powder is prepared according to the formulation in Table 15. Proteins (pea protein concentrate and fava bean protein isolate) are hydrated at 45-55% solids in 145-150 °F water. Then, the oils (high oleic safflower oil and canola oil), lecithin and fat soluble vitamin premix are added followed by the maltodextrin, sugar and fructooligosaccharides. Mineral and trace minerals as well as vitamins and nutrients are added. The mix is held at 140-145 °F at high agitation during mixing, then homogenized at 2000-3500 PSI and cooled to 40 °F. Vitamin C in 5% solution with a portion of potassium hydroxide and human lactoferrin in 10% solution along with N&A flavors are added to the cold mix. The mix is pasteurized at 170-180 °F for 60 seconds followed by spray-drying to a resulting moisture content of 2.5-4.0%.

The human lactoferrin can be added by dry-blending.

TABLE 15

| Ingredients | % Range |
|---|---|
| Maltodextrin | 35-40 |
| Sugar | 16-20 |
| Pea Protein Concentrate | 10-13 |
| High Oleic Safflower oil | 10-13 |
| Fava Bean Protein Concentrate | 10-13 |
| Canola oil | 4-5 |
| Fructooligosaccharides | 1.8-2.2 |
| Tricalcium Phosphate | 1.1-1.3 |
| Magnesium Chloride | 0.9-1.1 |
| Lecithin | 0.8-1.0 |
| Sodium Citrate | 0.4-0.6 |
| Potassium Citrate | 0.4-0.6 |
| Sodium Hydroxide | 0.4-0.6 |
| Magnesium Phosphate | 0.4-0.5 |
| Potassium hydroxide | 0.08-0.12 |
| Vitamin C | 0.04-0.06 |
| Human lactoferrin | 0.04-0.06 |
| Potassium Chloride | 0.04-0.06 |
| Natural & artificial flavors | 0.18-0.25 |
| Choline Chloride | 0.18-0.25 |
| Trace mineral premix | 0.15-0.18 |
| Potassium Phosphate | 0.14-0.17 |
| Water soluble vitamin premix | 0.05-0.09 |
| Fat soluble vitamin premix | 0.02-0.04 |
| Potassium Iodide | 0.00007-0.00009 |
| Moisture | 2.5-4.0 |

### Example 16

An immunity shot beverage with human lactoferrin was prepared using the formulation of Table 16. Human lactoferrin was mixed with water and stirred for 10 minutes. A high protein content fruit juice is selected, for example, guava, orange, blackberry, banana, peach, kiwi and so on. Alternatively, a protein source, such as, whey protein, can be included. An elderberry juice-based matrix was blended with the human lactoferrin solution for a few minutes and transferred to a plastic bottle (~60 mL). Blended samples were cooled to ~4 °C and high pressure process (HPP) treated at ~87,000 psi.

The bottles were tested for shelf stability.

TABLE 16

| Ingredient | % Range |
|---|---|
| Fruit Juice Blend | 99-99.999% |
| Human Lactoferrin | 0.001-1.0% |

### Example 17

A batch of liquid nutritional (ready-to-feed) composition is prepared using the formulation in Table 17. Reverse osmosis (RO) water is heated to 150-160 °F. Pea protein isolate, cellulose gel & gum, and tapioca starch are hydrated using high shear agitation. While maintaining high agitation, the oils (high oleic safflower oil and canola oil), lecithin, and fat soluble vitamin premix are added followed by the maltodextrin and sucralose. Individual minerals, the trace mineral premix, the water soluble vitamin premix and other nutrients are added. The mix is held at 140-145 °F at high agitation during mixing, then pasteurized at 185-190 °F for 30 seconds, homogenized at 2000-3500 PSI and cooled at 40 °F. Vitamin C in 5% solution with a portion of potassium hydroxide and human lactoferrin in 10% solution along with N&A flavors are added to the cold mix along with the residual RO water to bring the total solids to 20-30% w/w. The mix is UHT-treated and filled aseptically in 8 oz plastic bottle.

The composition is suitable for FODMAP (fermentable oligosaccharides, disaccharides, monosaccharides and polyols) diets.

TABLE 17

| Ingredients | % Range |
|---|---|
| Water | 70-80% |
| Maltodextrin | 5.0-10% |
| Pea Protein Isolate | 4.0-10% |
| High Oleic Safflower Oil | 2.0-4.0% |
| Canola Oil | 0.5-2.0% |
| Sodium Citrate | 0.1-0.5% |
| Tricalcium Phosphate | 0.1-0.5% |
| Magnesium Chloride | 0.1-0.5% |
| Lecithin | 0.1-0.5% |
| Potassium Hydroxide | 0.05-0.3% |
| Potassium Citrate | 0.05-0.3% |
| Sodium Hydroxide | 0.05-0.3% |
| Magnesium Phosphate | 0.05-0.3% |
| Human Lactoferrin | 0.01-1.0% |
| Potassium Phosphate | 0.01-0.1% |
| Vitamin C | 0.01-0.1% |
| Potassium Chloride | 0.01-0.1% |
| Natural & Artificial Flavors | 0.01-0.1% |
| Choline Chloride | 0.01-0.1% |
| Trace Mineral Premix | 0.01-0.1% |
| Water Soluble Vitamin Premix | 0.01-0.05% |
| Cellulose Gel and Gum | 0.01-0.05% |
| Tapioca Starch | 0.001-1.0% |
| Sucralose | 0.001-0.1% |
| Fat Soluble Vitamin Premix | 0.001-0.05% |
| Potassium Iodide | 0.00001-0.0001% |

### Example 18

A batch of milk-based infant formula was prepared according to the formulation in Table 18. Lactose was hydrated at 45-55% solids in 140-145 °F water, then proteins were added followed by the vegetable oil blend, ARA-DHA blend and fat soluble vitamin premix. Mineral and trace minerals as well as vitamins and nutrients were added to the mixture. The mix was held at 117-127 °F at high agitation during mixing, then homogenized at 2000-3500 PSI and pasteurized at 170-180 °F for 60 seconds. The mixture was cooled to 122-131 °F before spray-drying to a resulting moisture content of 1.5-3.5%. Probiotics and human lactoferrin were added to the base formulation via dry-blending.

The resulting infant formula powder was packaged. The powder was added to water to form a drinkable infant formula.

TABLE 18

| Ingredient | Item Code | % Range |
|---|---|---|
| Lactose | — | 50-53% |
| Vegetable Oil blend | — | 22-28% |
| Casein | — | 5.0-7.5% |
| Whey Protein Lipid Concentrate (MFGM) | — | 2.5-5.5% |
| Alpha-Lac enriched Whey Protein | — | 2.5-5.0% |
| Mineral Premix | XR73952000 | 1.0-3.5% |
| 2′-Fucosyllactose (HMO) | — | 1.5-2.0% |
| ARA-DHA Blend | — | 0.5-1.0% |
| Choline Bitartrate | — | 0.2-0.5% |
| Vitamin Premix | XR74360000 | 0.2-0.6% |
| Potassium Hydroxide | — | 0.1-0.3% |
| Ascorbic Acid | — | 0.1-0.3% |
| Potassium Bicarbonate | — | 0.05-0.12% |
| Trace Mineral Premix | XR73965000 | 0.02-0.07% |
| Nucleotide Premix | XR73202000 | 0.01-0.05% |
| Liquid Fat Soluble Premix | XR74558000 | 0.01-0.05% |
| Human Lactoferrin | — | 0.4-1.0% |
| Probiotic Microorganisms | — | 0.05-0.2% |
| Moisture | — | 1.5-3.5% |

### Example 19

A nutrition bar fortified with human milk oligosaccharides (HMO's) and human lactoferrin is prepared using the formulation in Table 19. Powder ingredients including HMO's powder, human lactoferrin and vitamin premix are mixed. Liquid components of binder syrup (maltitol syrup, chocolate liquor) are blended and heated to 150 °F. Dry components of the binder, such as, cocoa powder are mixed together and heated to 180 °F. Binder materials are added to blended powder ingredients and stirred using a spatula. Approximately 160 g of mixed sample is placed into a bar press mold and chilled in the freezer for 5-10 minutes.

TABLE 19

| Ingredients | % Range |
|---|---|
| Core Component Caramel | 25-35% |
| Soy Protein Crisps | 16.5-22.5% |
| Compound Coating Chocolate | 15-25% |
| Brown Rice Syrup | 5-10% |
| Caramel | 2-7% |
| rhLF | 0.1-3.0% |
| Human Milk Oligosaccharides | 1-5% |
| Casein | 0.5-3% |
| Tricalcium Phosphate | 0.5-3.0% |
| Chocolate Flavor | 0.5-3.0% |
| Vanilla Flavor | 0.5-3.0% |
| Maltitol Syrup | 0.1-5% |
| Glycerin | 0.1-3% |
| Cocoa Powder | 0.1-3.0% |
| Chocolate Liquor | 0.1-1.0% |
| Crystalline Fructose | 0.1-1.0% |
| Salt | 0.1-1.0% |
| Vitamin and Mineral Blend | 0.1-0.5% |
| Soy Lecithin | 0.01-0.5% |

### Example 20

A batch of milk-based infant formula with OPO is prepared according to the formulation in Table 20. Lactose is hydrated at 45-55% solids in 150-160 °F water, then proteins are added followed by the vegetable oil blend consisting of Sn2 palmitate fat (OPO), coconut oil, high oleic safflower or sunflower oil, soybean oil and sunflower or canola lecithin. ARA-DHA blend and fat soluble vitamin premix are added. Mineral and trace minerals as well as vitamins and nutrients are added. The mix is held at 140-145 °F at high agitation during mixing, then homogenized at 2000-3500 PSI and cooled at 40 °F. Nucleotide premix is added to the cold mix and agitated before pasteurization at 170-180 °F for 60 seconds followed by spray-drying to a resulting moisture content of 1.5-3.5%. Probiotics and human lactoferrin are added to the base formulation via dry-blending.

TABLE 20

| Ingredient | Item Code | % Range |
|---|---|---|
| Lactose | — | 45-49% |
| Whole Milk Powder | — | 14-17% |
| Sn2 Palmitate fat (OPO) | — | 6.0-9.0% |
| Whey Protein Concentrate 80% | — | 4.0-7.0% |
| Coconut Oil | — | 3.0-5.5% |
| High Oleic Safflower or Sunflower Oil | — | 3.0-5.5% |
| Soybean Oil | — | 3.0-5.5% |
| Alpha-Lac enriched Whey Protein | — | 1.5-4.0% |
| Mineral Premix | XR73983000 | 1.0-3.5% |
| 2′-Fucosyllactose (HMO) | — | 1.5-2.0% |
| ARA-DHA Blend | — | 0.5-1.0% |
| Choline Bitartrate | — | 0.2-0.5% |
| Vitamin Premix | XR74360000 | 0.2-0.6% |
| Sunflower or Canola Lecithin | — | 0.1-0.4% |
| Potassium Hydroxide | — | 0.1-0.3% |
| Ascorbic Acid | — | 0.1-0.3% |
| Potassium Bicarbonate | — | 0.05-0.12% |
| Trace Mineral Premix | XR73965000 | 0.02-0.07% |
| Nucleotide Premix | XR73202000 | 0.01-0.05% |
| Liquid Fat Soluble Premix | XR74558000 | 0.01-0.05% |
| Human Lactoferrin | — | 0.4-1.0% |
| Probiotic Microbes | — | 0.05-0.2% |
| Moisture | — | 1.5-3.5% |

### Example 21

A batch of prenatal formula powder (with DHA and choline) is prepared according to the formulation in Table 21. Proteins (milk protein concentrate and soy protein isolate) are hydrated at 45-55% solids in 145-150 °F water then the oils (high oleic safflower oil and canola oil), lecithin, DHA oil and fat soluble vitamin premix are added followed by the maltodextrin, sugar and fructooligosaccharides. Mineral and trace minerals as well as other vitamins and nutrients are added. The mix is held at 140-145 °F at high agitation during mixing, then homogenized at 2000-3500 PSI and cooled at 40 °F. Vitamin C in 5% solution with a portion of potassium hydroxide and human lactoferrin in 10% solution (the human lactoferrin can be added at another stage by dry blending) along with N&A flavors are added to the cold mix. The mix is pasteurized at 170-180 °F for 60 seconds followed by spray-drying to a resulting moisture content of 2.5-4.0%.

TABLE 21

| Ingredients | % Range |
|---|---|
| Water | 76-77 |
| Maldodextrin | 5.8-6.5 |
| Sugar | 4.0-4.5 |
| Milk Protein Concentrate | 2.5-3.0 |
| High Oleic Safflower oil | 2.5-3.0 |
| Soy Protein Isolate | 2.5-3.0 |
| Choline Bitartrate | 0.4-0.6 |
| Fructooligosaccharides | 0.4-0.6 |
| Canola oil | 0.2-0.4 |
| Tricalcium Phosphate | 0.2-0.4 |
| Magnesium Chloride | 0.2-0.3 |
| Lecithin | 0.1-0.2 |
| Sodium Citrate | 0.1-0.2 |
| Potassium Citrate | 0.08-0.15 |
| Sodium Hydroxide | 0.08-0.13 |
| Magnesium Phosphate | 0.08-0.13 |
| Natural & artificial flavors | 0.08-0.12 |
| Potassium hydroxide | 0.08-0.12 |
| DHA Oil | 0.05-0.08 |
| Vitamin C | 0.05-0.08 |
| Human lactoferrin | 0.04-0.07 |
| Potassium Chloride | 0.04-0.07 |
| Choline Chloride | 0.04-0.06 |
| Trace mineral premix | 0.03-0.04 |
| Potassium Phosphate | 0.03-0.04 |
| Water soluble vitamin premix | 0.015-0.021 |
| Cellulose gel and gum | 0.010-0.015 |
| Fat soluble vitamin premix | 0.005-0.010 |
| Carrageenan | 0.001-0.003 |
| Potassium Iodide | 0.00001-0.00003 |

### Example 22

Recombinant human lactoferrin (rhLF) produced in P. pastoris (K. phaffii) was tested to ascertain survivability of oral dosing forms through the digestive system. Apo and holo forms were tested to determine effect, if any, of iron loading. Native apo and holo human and bovine lactoferrin served as controls.

A simulated salivary fluid was prepared. The pH was 7. A sample of lactoferrin was added and the mixture incubated with mixing at 37 °C for 2 minutes. A sample was taken for SDS gel and Western blot testing for LF presence.

A simulated gastric chyme and fluid were prepared. The pH was 3. Porcine pepsin (Sigma) (2000 U/ml) was added. The simulated oral cavity sample was introduced into the simulated gastric fluid and the mixture incubated with mixing at 37 °C for 2 hours. A sample was taken for SDS gel and Western blot testing for LF presence.

A simulated intestinal fluid was prepared. Bile salts (Sigma) (10 mM), 100 U/ml of porcine trypsin (Sigma) and 25 U/m of porcine chymotrypsin (sigma) were added. The pH of the mixture was 7. The simulated gastric sample was introduced to the simulated intestinal fluid and the mixture incubated with mixing at 37 °C for 2 hours. A sample was taken for SDS gel and Western blot testing for LF presence.

Testing revealed intact protein and LF peptides for apo and holo forms of the enzymes after the gastric phase. LF peptides were detected following intestinal digestion. The holo forms yielded slightly greater amounts of peptides.

Recombinant human lactoferrin behaved similarly to human and bovine apo and holo forms of lactoferrin.

### Example 23

It was suggested allergens are pepsin resistant, Almond et al., supra, for example, bovine β-lactoglobin. Thus, rhLF was tested for level of susceptibility to pepsin.

A simulated gastric fluid (SGF) was prepared with a pH of 1-2 and 1600 U/ml porcine pepsin (Sigma). Holo (iron loaded) and apo forms of native bovine and human LF, and rhLF were tested.

After LF was added to the SGF, the mixture was incubated at 37 °C for 60 minutes, with samples taken at 0, 0.5, 2, 5, 10, 30 and 60 minutes to monitor course of digestion, if any, by SDS gel electrophoresis and Western blot.

Over the 60 minute period, pepsin was present in all samples indicating there was no auto or self-digestion.

All LF samples were digested totally by 2 minutes.

### Example 24

Immunogenicity of rhLF produced in P. pastoris (K. phaffii) was tested. Cord blood mononuclear cells (StemCell) from a female infant were exposed to rhLF and to native bovine LF (Sigma) and human LF obtained from human breast milk. Samples were tested with or without LPS (Sigma) stimulation. Levels of IL-6, IL-1β, IL-8, TNF-α, IL-10, IL12p70, MCP1/CCL-2, MIP-1α/CCL-3 and MIP-1β/CCL-4 were determined. None of the samples contained endotoxin.

Neither native human LF nor rhLF stimulated cytokine production, with or without LPS. On the other hand, the human immune cells reacted to the bovine protein.

All materials and methods disclosed and claimed herein can be made and executed without undue experimentation in light of the present disclosure. Ingredients or reagents of edible compositions disclosed herein are available commercially as food grade or drug grade compounds or can be made as provided in the art. While compositions and methods of the disclosure have been described in terms of certain embodiments, it will be apparent to those of skill in the art that variations may be applied to the methods and in the steps or in the sequence of steps of the methods described herein without departing from the concept, spirit and scope of the disclosure. More specifically, it will be apparent that certain agents which are both chemically and physiologically related may be substituted for agents described herein while same or similar results would be achieved. All such similar substitutes and modifications apparent to those skilled in the art are deemed to be within the spirit, scope and concept of the present disclosure.

What is claimed is:

1. An edible or potable composition comprising a protein and a recombinant human secretory protein.

2. The edible or potable composition of claim 1, wherein said secretory protein comprises a human milk protein.

3. The edible or potable composition of claim 1, wherein said recombinant human secretory protein comprises secretory IgA (sIgA), serum albumin, xanthine dehydrogenase, lactoferrin, lactoperoxidase, butyrophilin, lactadherin, adiponectin, β-casein, κ-casein, leptin, lysozyme or α-lactalbumin.

4. The edible or potable composition of claim 1, wherein said recombinant human secretory protein comprises lactoferrin.

5. The edible or potable composition of claim 1, wherein said secretory protein comprises one or more fucose, hybrid N-glycan, complex N-glycan, N-acetylgalactosamine, mannose or sialic acid.

6. The edible or potable composition of claim 1, further comprising 2′-fucosyllactose, lacto-N-neotetraose, 3-fucosyllactose, 6′-sialyllactose, difucosyllactose or lacto-N-tetraose.

7. The edible or potable composition of claim 1, further comprising one or more lipids.

8. The edible or potable composition of claim 1, further comprising one or more minerals.

9. The edible or potable composition of claim 1, further comprising one or more vitamins.

10. The edible or potable composition of claim 1, further comprising one or more nucleotides.

11. The edible or potable composition of claim 1, further comprising whey protein lipid concentrate, whole milk powder, whey protein concentrate, non-fat dry milk, milk protein concentrate or isolate, whey protein hydrolysate or an isolated protein.

12. The edible or potable composition of claim 1, further comprising high oleic sunflower oil, mid-oleic sunflower oil, safflower oil, coconut oil, low erucic rapeseed oil, sunflower oil, ascorbyl palmitate, mixed tocopherol concentrate, Mortierella alpina oil, alga oil, soybean oil or palm oil.

13. The edible or potable composition of claim 1, further comprising one or more of calcium, phosphorous, iodine, iron, magnesium, copper, zinc, manganese, chloride, potassium, sodium or selenium.

14. The edible or potable composition of claim 1, further comprising one or more of vitamin A, vitamin D, vitamin E, vitamin K, thiamine, riboflavin, pyridoxine, niacinamide, calcium pantothenate, vitamin B6, biotin, folic acid, pantothenic acid, vitamin B12, vitamin C, choline, inositol, and salts thereof.

15. The edible or potable composition of claim 1, further comprising a probiotic or a prebiotic.

16. The edible or potable composition of claim 1, wherein said recombinant human secretory protein is from a mammal cell, a bacterium or a fungus cell.

17. The edible or potable composition of claim 1, wherein said recombinant human secretory protein is from a yeast cell.

18. The edible or potable composition of claim 1, wherein said recombinant human secretory protein is from a cell of genus, Arxula, Aspergillus, Aurantiochytrium, Candida, Claviceps, Cryptococcus, Cunninghamella, Geotrichum, Hansenula, Kluyveromyces, Kodamaea, Komagataella, Leucosporidiella, Lipomyces, Mortierella, Ogataea, Pichia, Prototheca, Rhizopus, Rhodosporidium, Rhodotorula, Saccharomyces, Schizosaccharomyces, Tremella, Trichosporon, Wickerhamomyces or Yarrowia.

19. The edible or potable composition of claim 1, wherein said recombinant human secretory protein comprises one or more N-glycans found on a human protein.

20. The edible or potable composition of claim 1, comprising from about 0.01% to about 2% by weight of said recombinant human secretory protein.


SEQUENCE LISTING (4 sequences)

SEQ ID NO: 1 - protein, 710 aa, Homo sapiens
  REGION 1..710: note = misc_feature - Full length human lactoferrin
MKLVFLVLLFLGALGLCLAGRRRSVQWCAVSQPEATKCFQWQRNMRKVRGPPVSCIKRDSPIQCIQAIAENRADAVTLDGGFIYEAGLAPYKLRPVAAEVYGTERQPRTHYYAVAVVKKGGSFQLNELQGLKSCHTGLRRTAGWNVPIGTLRPFLNWTGPPEPIEAAVARFFSASCVPGADKGQFPNLCRLCAGTGENKCAFSSQEPYFSYSGAFKCLRDGAGDVAFIRESTVFEDLSDEAERDEYELLCPDNTRKPVDKFKDCHLARVPSHAVVARSVNGKEDAIWNLLRQAQEKFGKDKSPKFQLFGSPSGQKDLLFKDSAIGFSRVPPRIDSGLYLGSGYFTAIQNLRKSEEEVAARRARVVWCAVGEQELRKCNQWSGLSEGSVTCSSASTTEDCIALVLKGEADAMSLDGGYVYTAGKCGLVPVLAENYKSQQSSDPDPNCVDRPVEGYLAVAVVRRSDTSLTWNSVKGKKSCHTAVDRTAGWNIPMGLLFNQTGSCKFDEYFSQSCAPGSDPRSNLCALCIGDEQGENKCVPNSNERYYGYTGAFRCLAENAGDVAFVKDVTVLQNTDGNNNEAWAKDLKLADFALLCLDGKRKPVTEARSCHLAMAPNHAVVSRMDKVERLKQVLLHQQAKFGRNGSDCPDKFCLFQSETKNLLFNDNTECLARLHGKTTYEKYLGPQYVAGITNLKKCSTSPLLEACEFLRK

SEQ ID NO: 2 - protein, 691 aa, Homo sapiens
  REGION 1..691: note = misc_feature - Mature human lactoferrin
GRRRSVQWCAVSQPEATKCFQWQRNMRKVRGPPVSCIKRDSPIQCIQAIAENRADAVTLDGGFIYEAGLAPYKLRPVAAEVYGTERQPRTHYYAVAVVKKGGSFQLNELQGLKSCHTGLRRTAGWNVPIGTLRPFLNWTGPPEPIEAAVARFFSASCVPGADKGQFPNLCRLCAGTGENKCAFSSQEPYFSYSGAFKCLRDGAGDVAFIRESTVFEDLSDEAERDEYELLCPDNTRKPVDKFKDCHLARVPSHAVVARSVNGKEDAIWNLLRQAQEKFGKDKSPKFQLFGSPSGQKDLLFKDSAIGFSRVPPRIDSGLYLGSGYFTAIQNLRKSEEEVAARRARVVWCAVGEQELRKCNQWSGLSEGSVTCSSASTTEDCIALVLKGEADAMSLDGGYVYTAGKCGLVPVLAENYKSQQSSDPDPNCVDRPVEGYLAVAVVRRSDTSLTWNSVKGKKSCHTAVDRTAGWNIPMGLLFNQTGSCKFDEYFSQSCAPGSDPRSNLCALCIGDEQGENKCVPNSNERYYGYTGAFRCLAENAGDVAFVKDVTVLQNTDGNNNEAWAKDLKLADFALLCLDGKRKPVTEARSCHLAMAPNHAVVSRMDKVERLKQVLLHQQAKFGRNGSDCPDKFCLFQSETKNLLFNDNTECLARLHGKTTYEKYLGPQYVAGITNLKKCSTSPLLEACEFLRK

SEQ ID NO: 3 - protein, 142 aa, Homo sapiens
  REGION 1..142: note = misc_feature - Full length human alpha-lactalbumin
MRFFVPLFLVGILFPAILAKQFTKCELSQLLKDIDGYGGIALPELICTMFHTSGYDTQAIVENNESTEYGLFQISNKLWCKSSQVPQSRNICDISCDKFLDDDITDDIMCAKKILDIKGIDYWLAHKALCTEKLEQWLCEKL

SEQ ID NO: 4 - protein, 123 aa, Homo sapiens
  REGION 1..123: note = misc_feature - Mature human alpha-lactalbumin
KQFTKCELSQLLKDIDGYGGIALPELICTMFHTSGYDTQAIVENNESTEYGLFQISNKLWCKSSQVPQSRNICDISCDKFLDDDITDDIMCAKKILDIKGIDYWLAHKALCTEKLEQWLCEKL